(19) United States
(12) Patent Application Publication
(10) Pub. No.: US 2009/0233355 A1
(43) Pub. Date: Sep. 17, 2009

(54) Compositions and methods for culturing embryos and oocytes

(76) Inventor: Claire Trelfold Roberts, Hawthorndene (AU)

Correspondence Address: BIRCH STEWART KOLASCH & BIRCH, PO BOX 747, FALLS CHURCH, VA, 22040-0747, US

(21) Appl. No.: 11/989,304

(22) PCT Filed: Jul. 27, 2006
(86) PCT No.: PCT/AU2006/001042

§ 371 Date: Jan. 24, 2008

(30) Foreign Application Priority Data
Jul. 27, 2005 (AU) 2005903997

(51) Int. Cl. C12N 5/02; C12N 5/06

(52) U.S. Cl. 435/373; 435/325; 435/405; 435/384

(57) ABSTRACT

The present invention relates to an oocyte and/or embryo culture medium. The medium includes 0.0003 to 750 ng/ml IGF-II, or a variant or analogue thereof, and further includes either or both of 0.01 to 50 μg/ml plasminogen, or a variant or analogue thereof, and 0.01 to 50 μg/ml urokinase plasminogen activator, or a variant or analogue thereof.

This application claims priority from Australian Provisional Patent Application No. 2005903997 filed on 27 Jul. 2005, the contents of which are to be taken as incorporated herein by this reference.

### FIELD OF THE INVENTION

The present invention relates to compositions and methods for culturing embryos and/or oocytes.

### BACKGROUND OF THE INVENTION

A significant proportion of children in western countries are now born using assisted reproduction technologies, including the use of in vitro fertilization (IVF). IVF generally takes the form of stimulating the female to ovulate, contacting collected ova with sperm in vitro and introducing the resultant embryo into the uterus.

Despite considerable research and technical advances in the IVF field, the rate of successful pregnancy following IVF treatment is still quite low and is in the order of 15 to 25% per cycle. The poor success rate for IVF treatment is due in large part to an extraordinarily high rate of early embryonic loss due to impaired development and/or implantation failure.

Implantation of the embryo into the uterine endometrium and formation of the placenta is a highly coordinated process involving the interaction between maternal and embryonic cells. Not only must an embryo develop to the blastocyst stage before implantation may occur, but synchronized physiological preparation of the maternal endometrium for implantation must also proceed. Preparation of the endometrium for implantation is modulated by the cyclic secretion of 17β-estradiol and progesterone, which regulate growth factors, cytokines and adhesion molecules that alter the endometrial surface and open the implantation window. Prior to attachment to the endometrial epithelium, the zona pellucida surrounding the blastocyst is also lost.

Immediately after attachment, the trophoblast cell layer of the blastocyst proliferates rapidly and differentiates into an inner cytotrophoblastic layer and an outer multinucleated syncytiotrophoblastic mass. The syncytiotrophoblast then extends into the endometrial epithelium and invades the connective tissue. The blastocyst sinks beneath the endometrial surface, which is gradually repaired. At this point, nourishment of the embryo is obtained from the eroded maternal tissues and lacunar networks that form within the syncytiotrophoblast. Maternal blood moves in and out of these networks, thus establishing the uteroplacental circulation. Extensions of proliferating cytotrophoblast cells evaginate into the syncytiotrophoblast in various places and as such are the first stage in the development of the chorionic villi of the placenta.

After successful implantation and initiation of placentation, trophoblast cells undergo extensive proliferation and differentiation. There are two main pathways by which trophoblast differentiation may occur, namely, villous and extravillous. By days 13 to 14 of pregnancy, cytotrophoblast cells penetrate the layer of syncytiotrophoblast surrounding the early conceptus to form columns of extravillous cytotrophoblast cells. These contiguous cells form the cytotrophoblastic shell that is at the interface of the feto-maternal compartments. Extravillous trophoblast cells invade the decidua and migrate so that they penetrate and remodel maternal blood vessels in the uterine decidua to form the placenta.

It is now recognised that implantation failure is not only responsible at least in part for the poor rates of success of IVF treatment, but improper implantation also has a number of consequences in patients with both normal and assisted pregnancies, including spontaneous miscarriage, pre-eclampsia, intrauterine growth restriction (also known as fetal growth restriction), pre-term birth and placental abruption.

It is also becoming increasingly apparent that the current methods for culturing oocytes and embryos in vitro for assisted reproduction technologies may have adverse effects on the development of the embryo, and may also affect the ability of the embryo to implant, ultimately affecting pregnancy outcome. Indeed, critical evaluation of the deficiencies of in vitro culture now appears essential in view of recent epidemiological studies that demonstrate growth impairment and an increased likelihood of major birth defects in children born after IVF.

Accordingly, there is a need for new media and methods for culturing oocytes and embryos. In particular, there is a need for new media and methods for culturing oocytes and embryos to improve the ability of embryos to successfully develop and/or implant into uterine endometrium. The present invention relates to an improved oocyte and embryo culture medium.

A reference herein to a patent document or other matter which is given as prior art is not to be taken as an admission that that document or matter was known or that the information it contains was part of the common general knowledge as at the priority date of any of the claims.

### SUMMARY OF THE INVENTION

The present invention provides an oocyte and/or embryo culture medium, the medium including 0.0003 to 750 ng/ml IGF-II, or a variant or analogue thereof, the medium further including either or both of 0.01 to 50 μg/ml plasminogen, or a variant or analogue thereof, and 0.01 to 50 μg/ml urokinase plasminogen activator, or a variant or analogue thereof.

The present invention also provides a method of culturing an oocyte and/or embryo, the method including the step of exposing the oocyte or embryo to a culture medium including 0.0003 to 750 ng/ml IGF-II, or a variant or analogue thereof, the method further including the step of exposing the oocyte or embryo to either or both of 0.01 to 50 μg/ml plasminogen, or a variant or analogue thereof, and 0.01 to 50 μg/ml urokinase plasminogen activator, or a variant or analogue thereof.

The present invention also provides a combination product including the following components:
an oocyte and/or embryo culture medium;
IGF-II, or a variant or an analogue thereof; and either or both of plasminogen, or a variant or analogue thereof, and
urokinase plasminogen activator, or a variant or an analogue thereof;
   wherein the components are provided in a form for addition of IGF-II, and either or both of plasminogen and urokinase plasminogen activator, to the culture medium so as to produce a culture medium including 0.0003 to 750 ng/ml IGF-II (or a variant or an analogue thereof), and either or both of 0.01 to 50 μg/ml plasminogen (or a variant or an analogue thereof) and 0.01 to 50 μg/ml urokinase plasminogen activator (or a variant or an analogue thereof).

The present invention also provides a composition when used for exposure to an isolated embryo or an isolated oocyte, the composition including 0.0003 to 750 ng/ml IGF-II, or a variant or analogue thereof, the composition further including either or both of 0.01 to 50 μg/ml plasminogen, or a variant or analogue thereof, and 0.01 to 50 μg/ml urokinase plasminogen activator, or a variant or analogue thereof.

The present invention also provides a method of assisted reproduction involving an oocyte or embryo, the method including the step of culturing the oocyte or embryo in a medium including 0.0003 to 750 ng/ml IGF-II, or a variant or analogue thereof, the method further including the step of exposing the oocyte or embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of in vitro fertilization of an oocyte, the method including the step of culturing the oocyte, or an embryo produced from the oocyte, in a medium including 0.0003 to 750 ng/ml IGF-II, or a variant or analogue thereof, the method further including the step of exposing the oocyte or embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of promoting implantation of an isolated embryo into uterine endometrium, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of promoting implantation into uterine endometrium of an embryo produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of culturing an isolated early stage embryo to blastocyst stage, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of maturing an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the likelihood of failure of an isolated embryo to implant into uterine endometrium, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the likelihood of implantation failure into uterine endometrium of an embryo produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of improving placental development for an isolated embryo implanted into uterine endometrium, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of improving placental development for an embryo implanted into uterine endometrium, the embryo being produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of improving placental growth and/or function for an isolated embryo implanted into uterine endometrium, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue, thereof.

The present invention also provides a method of improving placental growth and/or function for an embryo implanted into uterine endometrium, the embryo being produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the likelihood of miscarriage in a subject with an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the likelihood of miscarriage in a subject with an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the extent and/or likelihood of pre-eclampsia in a subject with an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the extent and/or likelihood of pre-eclampsia in a subject with an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the extent and/or likelihood of intrauterine growth restriction in a subject with an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the extent and/or likelihood of intrauterine growth restriction in a subject with an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the likelihood of pre-term delivery of a fetus produced from an isolated embryo introduced into a subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the likelihood of pre-term delivery of a fetus resulting from an embryo produced from an isolated oocyte introduced into a subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of increasing the likelihood that a fetus is carried to term or near term in a subject, the fetus being produced from an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of increasing the likelihood that a fetus is carried to term or near term in a subject, the fetus being produced from an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of normalising the length of the gestation period of a fetus carried by a subject, the fetus being produced from an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of normalising the length of the gestation period of a fetus carried by a subject, the fetus being produced from an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the extent and/or likelihood of abruption of a placenta formed by implantation of an isolated embryo into uterine endometrium, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides a method of reducing the extent and/or likelihood of abruption of a placenta formed by implantation into uterine endometrium of an embryo produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides an intravaginal composition, the composition including IGF-II, or a variant or an analogue thereof, and either or both of plasminogen, or a variant or an analogue thereof, and urokinase plasminogen activator, or a variant or an analogue thereof.

The present invention also provides a method of promoting implantation of an embryo into uterine endometrium in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention also provides a method of promoting placental development in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention provides a method of improving placental growth and/or function in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention also provides a method of reducing the likelihood of miscarriage occurring in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention also provides a method of reducing the extent and/or likelihood of pre-eclampsia occurring in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention also provides a method of reducing the extent and/or likelihood of intrauterine growth restriction occurring in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention also provides a method of reducing the likelihood of pre-term labour occurring in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention also provides a method of normalising the length of the gestation period of a fetus carried by a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention also provides a method of increasing the likelihood that a fetus is carried to term or near term, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention also provides a method of reducing the extent and/or likelihood of placental abruption occurring in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention arises from the recognition that improved media for the culture of oocytes and/or embryos may be produced by including in the medium IGF-II (or a variant or an analogue thereof) at a concentration in the range from 0.0003 to 750 ng/ml IGF-II, and also including in the medium either or both of plasminogen (or a variant or an analogue thereof) at a concentration in the range from 0.01 to 50 μg/ml plasminogen and urokinase plasminogen activator (or a variant or an analogue thereof) at a concentration in the range from 0.01 to 50 μg/ml.

It is also anticipated that exposing an embryo to IGF-II and either or both of plasminogen and urokinase plasminogen activator, will lead to an improvement in the ability of an embryo to implant into uterine endometrium.

This effect is also likely to extend to an unfertilized oocyte, namely, that exposing an oocyte to IGF-II and either or both of plasminogen and urokinase plasminogen activator, will lead to an improvement in an embryo produced from the oocyte to implant into uterine endometrium.

It is also anticipated that exposing an embryo to IGF-II and either or both of plasminogen and urokinase plasminogen activator, will lead to an improvement in the development of an embryo to the blastocyst stage.

Various terms that will be used throughout the specification have meanings that will be well understood by a skilled addressee. However, for ease of reference, some of these terms will now be defined.

The term “culture medium” as used throughout the specification is to be understood to mean the liquid environment in which in an oocyte or embryo is maintained and/or propagated.

The term “isolated” as used throughout the specification in relation to oocytes and embryos is to be understood to mean that the oocyte or embryo has at some time been removed or purified (at least partially) from its natural environment. An example of an isolated embryo is an embryo produced in vitro using an assisted reproduction technology or an embryo isolated from a subject. An example of an isolated oocyte is an oocyte isolated from a subject as part of a follicle, a cumulus oocyte complex, or a denuded oocyte.

It will be appreciated that the term “isolated” also extends to an isolated embryo or isolated oocyte that is introduced into a subject. For example, an embryo or oocyte may be isolated, produced and/or manipulated in vitro, and then subsequently introduced into a subject and treated with IGF-II, and either or both of plasminogen and uPA in vivo.

The term “likelihood” as used throughout the specification is to be understood to mean the increased probability that a specific event may occur in a subject, as compared to the probability that the same event may occur in another subject with similar risk factors. Generally, a subject with an increased probability that a specific event may occur will be identified by one or more of the following means: (i) having a known predisposition to the event; (ii) based on familial history of the event; (iii) based on clinical assessment; and (iv) based on a suitable diagnostic test. It will also be appreciated that in the case where the probability that a specific event occurring in the subject is due to paternal factors contributed by the male partner in the couple, the male partner may be identified by one or more of the same assessments.

The term “variant” as used throughout the specification is to be understood to mean an amino acid sequence that is altered by one or more amino acids. The variant may have “conservative” changes, wherein a substituted amino acid has similar structural or chemical properties to the replaced amino acid (e.g., replacement of leucine with isoleucine). A variant may also have “non-conservative” changes (e.g., replacement of a glycine with a tryptophan) or a deletion and/or insertion of one or more amino acids.

The term “analogue” as used throughout the specification is to be understood to mean a molecule having similar structural, regulatory, or biochemical functions as that of the reference molecule, and includes a biologically active fragment of the reference molecule.

The term “subject” as used throughout the specification is to be understood to mean a female human, a female mammal including a primate, a livestock animal (eg. a horse, a cow, a sheep, a pig, a goat), a companion animal (eg. a dog, a cat), a laboratory test animal (eg. a mouse, a rat, a guinea pig), or an animal of veterinary significance.

The term “assisted reproduction” as used throughout the specification is to be understood to mean any technique involving the production of an embryo capable of implantation, including a technique using an oocyte or embryo cultured in vitro (for example in vitro maturation of an oocyte), in vitro fertilization (IVF; aspiration of an oocyte, fertilization in the laboratory and transfer of the embryo into a recipient), gamete intrafallopian transfer (GIFT, placement of oocytes and sperm into the fallopian tube), zygote intrafallopian transfer (ZIFT; placement of fertilized oocytes into the fallopian tube), tubal embryo transfer (TET; the placement of cleaving embryos into the fallopian tube), peritoneal oocyte and sperm transfer (POST; the placement of oocytes and sperm into the pelvic cavity), intracytoplasmic sperm injection (ICSI), testicular sperm extraction (TESE), microsurgical epididymal sperm aspiration (MESA), nuclear transfer, expansion from a totipotent stem cell and parthenogenic activation. Methods of assisted reproduction are known in the art.

### General Description of the Invention

As discussed above, in one embodiment the present invention provides an oocyte and/or embryo culture medium, the medium including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The culture medium is suitable for all stages of development of an oocyte and/or embryo.

The culture medium is also suitable for use for in vitro maturation of an oocyte, in vitro fertilization of an oocyte, culturing an embryo to the blastocyst stage, and as a transfer medium.

During pregnancy, particularly in humans, IGF-II is produced by placental cytotrophoblast cells and promotes invasion of cytotrophoblast cells cells into the decidua and its vasculature, with IGF-II being most abundantly expressed at the invasive front.

TGF-β1 has an opposing action to IGF-II, promoting terminal differentiation in these cells, thus inhibiting their migratory behaviour. Active TGF-β1 is formed from latent TGF-β1 by the urokinase plasminogen activator (uPA) system. In this system, plasmin converts latent TGF-β1 into active TGF-β1 by the catalytic action of plasmin generated in a proteolytic cascade when bound in its inactive form, plasminogen, to the complex formed by the simultaneous association of urokinase plasminogen activator (uPA), uPA receptor, plasminogen and latent TGF-β1 with the cation independent mannose-6-phosphate or IGF-II (CIM6P/IGF2) receptor expressed on the surface of cytotrophoblast cells.

IGF-II and latent TGF-β1, the inactive precursor of TGF-β1, compete for binding to the CIM6P/IGF2 receptor expressed on the surface of cytotrophoblasts. IGF-II prevents latent TGF-β1 binding to the CIM6P/IGF2 receptor and as such prevents activation of latent TGF-β1 into active TGF-β1. Cells that produce sufficient amounts of IGF-II cannot convert latent TGF-β1 into its active form by the uPA system. Thus, competition between IGF-II and latent TGF-β1 for binding to the CIMP6/IGF2 receptor underlies the action of IGF-II in regulating the ability of cytotrophoblasts to undergo migratory or non-migratory behaviour.

As discussed previously herein, the present invention is based on the recognition that improved media for the culture of oocytes and/or embryos may be produced by including in a medium IGF-II (or a variant or an analogue thereof) at a concentration in the range from 0.0003 to 750 ng/ml IGF-II, and also including in the medium either or both of plasminogen (or a variant or an analogue thereof) at a concentration in the range from 0.01 to 50 μg/ml and urokinase plasminogen activator (or a variant or analogue thereof) at a concentration of 0.01 to 50 μg/ml.

In one embodiment, the medium includes IGF-II and plasminogen. In another embodiment, the medium includes IGF-II and uPA. In another embodiment, the medium includes IGF-II, plasminogen and uPA.

In particular, it is anticipated that exposing an embryo to IGF-II and either or both of plasminogen and urokinase plasminogen activator will lead to an improvement in the ability of an embryo to implant into uterine endometrium. This effect is also likely to extend to an unfertilized oocyte, namely, that exposing an oocyte to IGF-II and either or both of plasminogen and urokinase plasminogen activator, will also lead to an improvement in an embryo produced from the oocyte to implant into uterine endometrium.

Accordingly, in another embodiment the present invention provides a method of culturing an oocyte and/or embryo, the method including the step of exposing the oocyte and/or embryo to a culture medium including 0.0003 to 750 ng/ml IGF-II, or a variant or analogue thereof, the method further including the step of exposing the oocyte and/or embryo to either or both of 0.01 to 50 μg/ml plasminogen, or a variant or analogue thereof, and 0.01 to 50 μg/ml urokinase plasminogen activator, or a variant or analogue thereof.

Methods for the culturing of oocytes and embryos are known in the art.

In one embodiment, the exposure to plasminogen and/or uPA is also by exposure to a medium including these agents.

It will be appreciated that IGF-II, and either or both of plasminogen and uPA may also be used in a combination product as a culture medium supplement for culturing an embryo and/or oocyte.

Accordingly, in another embodiment the present invention provides a combination product including the following components:
an oocyte and/or embryo culture medium;
IGF-II, or a variant or an analogue thereof; and either or both of plasminogen, or a variant or analogue thereof, and
urokinase plasminogen activator, or a variant or an analogue thereof;
   wherein the components are provided in a form for addition of IGF-II, and either or both of plasminogen and urokinase plasminogen activator, to the culture medium so as to produce a culture medium including 0.0003 to 750 ng/ml IGF-II (or a variant or an analogue thereof), and either or both of 0.01 to 50 μg/ml plasminogen (or a variant or an analogue thereof) and 0.01 to 50 μg/ml urokinase plasminogen activator (or a variant or an analogue thereof).

The culture medium, IGF-II, plasminogen and uPA in the various combination products of the present invention may be packaged separately in suitable containers (typically sterilized) such as ampoules, bottles, or vials, either in multi-use or in unit forms. The containers may be hermetically sealed after being filled. IGF-II, plasminogen and uPA may be in isolated form, in purified or semi-purified form, or in recombinant form, and may contain additional additives for the stability and/or use of the proteins. Methods for packaging the various components are known in the art.

It is anticipated that exposure of an oocyte or embryo to IGF-II, and either or both plasminogen and urokinase plasminogen activator will not only lead to an improvement in implantation and to an improvement in the outcomes for assisted reproduction technologies, but will also lead to reductions in implantation failure, miscarriage, including recurrent spontaneous miscarriage, pre-eclampsia, intrauterine growth restriction, pre-term birth and placental abruption.

In addition, it is also anticipated that an improvement in implantation will lead to an improvement in post natal outcomes by improving placental development and/or reducing the risk and/or likelihood of pregnancy complications.

In one embodiment, the embryo in the various relevant forms of the present invention is a human embryo or a mammalian embryo. Examples of suitable mammals include a primate, a livestock animal (eg. a horse, a cow, a sheep, a pig, a goat), a companion animal (eg. a dog, a cat), a laboratory test animal (eg. a mouse, a rat, a guinea pig). In one embodiment, the embryo is a human embryo.

In this regard, it will be appreciated that the term “embryo” means any cell or group of cells that result from fertilization, parthenogenic activation, nuclear transfer or expansion from a totipotent stem cell, and which has the capacity to form a blastocyst capable of implantation. In this regard, the term includes a fertilized oocyte, a zygote, an oocyte with a transferred nucleus, or one or more totipotent cells.

In one embodiment, the oocyte in the various forms of the present invention is a human oocyte or a mammalian oocyte. Examples of suitable mammals include a primate, a livestock animal (eg. a horse, a cow, a sheep, a pig, a goat), a companion animal (eg. a dog, a cat), a laboratory test animal (eg. a mouse, a rat, a guinea pig). In one embodiment, the oocyte is a human oocyte.

In this regard, the oocyte may be, for example, an oocyte that is part of a follicle, part of a cumulus oocyte complex (COC) or may be a denuded oocyte.

The culture medium of the present invention is suitable not only for use in humans, but also for culturing oocytes and embryos from animals. Thus, not only does the present invention have application to assisted reproduction technologies in humans, but it is also applicable to assisted reproduction techniques in non-human animals, and other technologies of producing embryos in non-human animals, such as the use of parthenogenic activation, nuclear transfer and the use of totipotent stem cells.

Accordingly, the present invention not only contemplates embryos and oocytes cultured in the medium according to the present invention, but also non-human animals produced from oocytes and embryos cultured in the medium according to the present invention.

The present invention is also suitable for the production of compositions for culturing isolated embryos and/or isolated oocytes.

Accordingly, in another embodiment the present invention provides a composition for exposure to an isolated oocyte and/or an isolated embryo, the composition including 0.0003 to 750 ng/ml IGF-II, or a variant or analogue thereof, the composition further including either or both of 0.01 to 50 μg/ml plasminogen, or a variant or analogue thereof, and 0.01 to 50 μg/ml urokinase plasminogen activator, or a variant or analogue thereof.

Methods are known in the art for collecting oocytes from suitable donor females and fertilizing the oocytes in vitro. For example, human in vitro fertilization may be performed as described in Textbook of Assisted Reproduction: Laboratory and Clinical Perspectives (2003) Editors Gardner, D. K., Weissman, A., Howles, C. M., Shoham, Z. Martin Dunits Ltd, London, UK. In vitro fertilization of cattle may be performed as described in Gordon, I. (2003) Laboratory Production of Cattle Embryos 2^{nd} Edition CABI Publishing, Oxon, UK.

Methods are also known in the art for producing an embryo by means other than the fertilization of an oocyte.

For example, methods are also known in the art for parthenogenically activating embryos in vitro. For example, a method of producing mice offspring by parthenogenesis is as described in Kono et al. (2004) Nature 428(6985):809-811.

Methods for producing embryos by nuclear transfer are known in the art (for example Campbell et al. (1995) Theriogenology 43: 181 (1995); Collas et al. (1994) Mol. Reprod. Dev. 38: 264-267; Keefer et al. (1994) Biol. Reprod. 50: 935-939; and Sims et al. (1993) Proc. Natl. Acad. Sci., USA 90: 6143-6147.

The IGF-II in the various embodiments of the present invention may be a recombinant form of the molecule, a substantially purified form of the molecule or a partially purified form of the molecule. Methods for the production and/or purification of IGF-II are known in the art.

It will also be appreciated that the IGF-II may be derived from any suitable species, and generally will be IGF-II derived from the same species as that of the target embryo or oocyte.

A variant of IGF-II is a form of the IGF-II molecule with an amino acid sequence in which one or more native amino acids is altered. In one embodiment, the variant has greater than 75% homology at the amino acid level with native IGF-II. In a further embodiment, the variant has greater than 90% homology with native IGF-II. In a further embodiment, the variant has greater than 95% homology with native IGF-II.

An analogue of IGF-II is a molecule having similar structural (ie a structural analogue), regulatory (ie a regulatory analogue), or biochemical functions (ie a functional analogue) as that of IGF-II, and includes a biologically active fragment of IGF-II. For example, the analogue may be an oligopeptide, a polypeptide, or an antibody that has a similar binding capacity as IGF-II to the CIMP6/IGF2 receptor.

Examples of molecules that may function as an analogue of IGF-II include proteins, polypeptides, polysaccharides, glycoproteins, hormones, receptors, lipids, small molecules, drugs, metabolites, cofactors, transition state analogues, and aptamers.

In one embodiment, the concentration of IGF-II in the medium (and thus the concentration of IGF-II exposed to an oocyte or embryo) is 0.003 to 750 ng/ml, and in a another embodiment is 1 to 750 ng/ml, and in another embodiment, 0.003 to 375 ng/ml, and in another embodiment 1 to 375 ng/ml, and in another embodiment 7.5 to 375 ng/ml IGF-II. A suitable concentration of IGF-II is 185 ng/ml. 375 ng/ml IGF-II corresponds to a concentration of approximately 50 nM.

The plasminogen in the various embodiments of the present invention may be a recombinant form of the molecule, a substantially purified form of the molecule or a partially purified form of the molecule. Methods for the production and/or purification of plasminogen are known in the art.

It will also be appreciated that the plasminogen may be derived from any suitable species and generally will be plasminogen derived from the same species as that of the target embryo or oocyte.

A variant of plasminogen is a form of the plasminogen molecule with an amino acid sequence in which one or more native amino acids is altered. In one embodiment, the variant has greater than 75% homology at the amino acid level with native plasminogen. In a further embodiment, the variant has greater than 90% homology with native plasminogen. In a further embodiment, the variant has greater than 95% homology with native plasminogen.

An analogue of plasminogen is a molecule having similar structural (ie a structural analogue), regulatory (ie a regulatory analogue), or biochemical functions (ie a functional analogue) as that of plasminogen, and includes a biologically active fragment of plasminogen.

Examples of molecules that may function as an analogue of plasminogen include proteins, polypeptides, polysaccharides, glycoproteins, hormones, receptors, lipids, small molecules, drugs, metabolites, cofactors, transition state analogues, and aptamers.

In one embodiment, the concentration of plasminogen in the medium (and thus the concentration of plasminogen exposed to an oocyte or embryo) is 1-20 μg/ml. A suitable concentration of plasminogen is 10 μg/ml.

As discussed previously, the medium may also include 0.01 to 50 μg/ml urokinase plasminogen activator, or a variant or analogue thereof.

In this regard, the inclusion of urokinase plasminogen activator in the culture medium in one embodiment is anticipated to also improve the ability of an embryo to implant into uterine endometrium.

The urokinase plasminogen activator in the various embodiments of the present invention may be a recombinant form of the molecule, a substantially purified form of the molecule or a partially purified form of the molecule. Methods for the production and/or purification of urokinase plasminogen activator are known in the art.

It will also be appreciated that the urokinase plasminogen activator may be derived from any suitable species and generally will be urokinase plasminogen activator derived from the same species as that of the target embryo or oocyte.

A variant of urokinase plasminogen activator is a form of the urokinase plasminogen activator molecule with an amino acid sequence in which one or more native amino acids is altered. In one embodiment, the variant has greater than 75% homology at the amino acid level with native urokinase plasminogen activator. In a further embodiment, the variant has greater than 90% homology with native urokinase plasminogen activator. In a further embodiment, the variant has greater than 95% homology with native urokinase plasminogen activator.

An analogue of urokinase plasminogen activator is a molecule having similar structural (ie a structural analogue), regulatory (ie a regulatory analogue), or biochemical functions (ie a functional analogue) as that of urokinase plasminogen activator, and includes a biologically active fragment of urokinase plasminogen activator.

Examples of molecules that may function as an analogue of urokinase plasminogen activator include proteins, polypeptides, polysaccharides, glycoproteins, hormones, receptors, lipids, small molecules, drugs, metabolites, cofactors, transition state analogues, and aptamers.

In one embodiment, the concentration of urokinase plasminogen activator in the medium (and thus the concentration of urokinase plasminogen activator exposed to an oocyte or embryo) is 1-20 μg/ml. A suitable concentration of urokinase plasminogen activator is 5 μg/ml.

The medium is suitable for culturing oocytes and/or embryos that are used for assisted reproduction technologies.

Accordingly, in another embodiment the present provides a method of assisted reproduction involving an oocyte or embryo, the method including the step of culturing the oocyte or embryo in a medium including 0.0003 to 750 ng/ml IGF-II, or a variant or analogue thereof, the method further including the step of exposing the oocyte or embryo to either or both of 0.01 to 50 μg/ml plasminogen, or a variant or analogue thereof, and 0.01 to 50 μg/ml urokinase plasminogen activator, or a variant or analogue thereof.

For example, the present invention may be used in an in vitro fertilization technique.

Accordingly, in another embodiment the present invention provides a method of in vitro fertilization of an oocyte, the method including the step of culturing the oocyte, or an embryo produced from the oocyte, in a medium including 0.0003 to 750 ng/ml IGF-II, or a variant or analogue thereof, the method further including the step of exposing the oocyte or embryo to either or both of 0.01 to 50 μg/ml plasminogen, or a variant or analogue thereof, and 0.01 to 50 μg/ml urokinase plasminogen activator, or a variant or analogue thereof.

The medium is also anticipated to be suitable for the maturation of an oocyte in vitro.

Accordingly, in another embodiment the present invention provides an oocyte in vitro maturation medium, the medium including 0.0003 to 750 ng/ml IGF-II, or a variant or analogue thereof, the medium further including either or both of 0.01 to 50 μg/ml plasminogen, or a variant or analogue thereof, and 0.01 to 50 μg/ml urokinase plasminogen activator, or a variant or analogue thereof.

A suitable period of time for incubation of the oocyte for maturation is 1-3 days.

The present invention is also anticipated to be suitable for the production of a composition for maturation of an isolated oocyte.

Accordingly, in another embodiment the present invention provides a composition for in vitro maturation of an isolated oocyte and/or an isolated embryo, the composition including 0.0003 to 750 ng/ml IGF-II, or a variant or analogue thereof, the composition further including either or both of 0.01 to 50 μg/ml plasminogen, or a variant or analogue thereof, and 0.01 to 50 μg/ml urokinase plasminogen activator, or a variant or analogue thereof.

The present invention also provides a combination product for in vitro maturation of an oocyte. A combination product including culture medium, IGF-II, and either or both of plasminogen and uPA is as previously discussed.

Culturing of oocytes in the in vitro maturation medium is also anticipated to produce an embryo that has an improved capacity to develop to the blastocyst stage, an improved capacity to implant, and also to increase the proportion of successful pregnancies that result from introduction of the embryo into a suitable subject.

Thus, the present invention may also be used, for example, to culture oocytes prior to fertilization for use in assisted reproduction technologies in humans and animals.

Accordingly, in another embodiment the present invention provides a method of maturing an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention may also be used to culture oocytes at any time prior to, during and/or after, nuclear transfer.

The isolated oocyte may be an oocyte in vitro, or alternatively, an isolated oocyte that has been introduced into a subject. In this regard, it will be understood that the present invention not only contemplates the exposure of an isolated oocyte in vitro to IGF-II and either or both of plasminogen and uPA, but also an isolated oocyte introduced into a subject and subsequently exposed to IGF-II and either or both of plasminogen and uPA.

The present invention also contemplates oocytes matured according to this form of the present invention, and embryos and non-human animals produced from the matured oocyte following fertilization.

The medium of the present invention is also suitable for culturing an embryo to the blastocyst stage. In this regard, it is also anticipated that the culture medium provides an improved medium for the development of an embryo.

Accordingly, in another embodiment the present invention provides a medium for culturing an early stage embryo to blastocyst stage, the medium including 0.0003 to 750 ng/ml IGF-II, or a variant or analogue thereof, the medium further including either or both of 0.01 to 50 μg/ml plasminogen, or a variant or analogue thereof, and 0.01 to 50 μg/ml urokinase plasminogen activator, or a variant or analogue thereof.

It is anticipated that the medium is suitable for increasing the proportion of early embryos that develop to the blastocyst stage and/or increasing the proportion of successful pregnancies that result from introduction of the embryo into a suitable subject. Thus, as described previously, the present invention may be used, for example, to culture oocytes and embryos in vitro for use in assisted reproduction technologies.

This is in part likely to be due to the fact that culturing the embryo in the medium of the present invention will improve development of the embryo and/or implantation of the embryo into uterine endometrium, and thereby (i) improve placental development; (ii) reduce the extent and/or likelihood of miscarriage, placental abruption, pre-eclampsia, including the extent and/or likelihood of recurrent spontaneous miscarriage (recurrent spontaneous abortion); (iii) reduce the extent and/or likelihood of intrauterine growth restriction; (iv) reduce the likelihood of pre-term birth or labour occurring; and (v) normalise the length of the gestation period of a fetus.

A suitable period of time for incubation of the embryo in the culture medium is from 2 to 5 days. Transfer ready blastocysts are embryos developed to the stage where a blastocoelic cavity is clearly evident and comprises greater than 50% of the volume of the embryo. This stage would in the in vivo situation normally be achieved 4-5 days after fertilisation, soon after the embryo has traversed the fallopian tube and arrives in the uterus.

Accordingly, in another embodiment the present invention provides a method of culturing an isolated early stage embryo to blastocyst stage, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The isolated embryo in the various relevant embodiments of the present invention may be an embryo in vitro, or alternatively, an isolated embryo that has been introduced into a subject. In this regard, it will be understood that the present invention not only contemplates the exposure of an isolated early stage embryo in vitro to IGF-II and either or both of plasminogen and uPA, but also an isolated early stage embryo introduced into a subject and subsequently exposed to IGF-II and either or both of plasminogen and uPA.

The present invention also provides an embryo cultured according to this form of the present invention, and non-human animals produced from embryos cultured according to this form of the present invention.

The present invention is also suitable for the production of a composition for culturing an isolated early stage embryo to the blastocyst stage.

Accordingly, in another embodiment the present invention provides a composition for exposure to an isolated early stage embryo for culturing the embryo to blastocyst stage, the composition including 0.0003 to 750 ng/ml IGF-II, or a variant or analogue thereof, the composition further including either or both of 0.01 to 50 μg/ml plasminogen, or a variant or analogue thereof, and 0.01 to 50 μg/ml urokinase plasminogen activator, or a variant or analogue thereof.

The present invention also provides a combination product for culturing an early stage embryo to blastocyst stage. A combination product including culture medium, IGF-II, and either or both of plasminogen and uPA is as previously discussed.

The medium is also suitable as an embryo transfer medium.

Accordingly, in another embodiment the present invention provides a medium for transfer of an isolated embryo into a subject, the medium including 0.0003 to 750 ng/ml IGF-II, or a variant or analogue thereof, the medium further including either or both of 0.01 to 50 μg/ml plasminogen, or a variant or analogue thereof, and 0.01 to 50 μg/ml urokinase plasminogen activator, or a variant or analogue thereof.

In another embodiment, the present invention provides a method of transferring an isolated embryo into a subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention is also suitable for the production of a composition for transferring an isolated embryo into a suitable female recipient.

Accordingly, in another embodiment the present invention provides a composition for exposure to an isolated embryo for transfer of the embryo into a subject, the composition including 0.0003 to 750 ng/ml IGF-II, or a variant or analogue thereof, the composition further including either or both of 0.01 to 50 μg/ml plasminogen, or a variant or analogue thereof, and 0.01 to 50 μg/ml urokinase plasminogen activator, or a variant or analogue thereof.

The present invention also provides a combination product for transfer of an embryo into a subject. A combination product including culture medium, IGF-II, and either or both of plasminogen and uPA is as previously discussed.

Suitable base media for the various embodiments of the present invention include HTF medium, Whittinghams T6 medium, Hams F10, Earles solution, IVF50 (Scandinavian IVF Science), S2 (Scandinavian IVF Science), G1.2 (Scandinavian IVF Science) and G2.2 (Scandinavian IVF Science).

In this regard, although oocytes and embryos may generally be cultured in many different types of media, the medium of the present invention is anticipated to provide an improvement to the implantation into uterine endometrium of embryos cultured in the medium. Although most media will support some level of development of embryos, they will not provide embryos that are developmentally competent and/or suitable for development and/or implantation. For example, media such as RPMI 1640 medium only support very limited embryo development, and as such would not be suitable for the development of embryos for implantation.

In one embodiment, the medium is serum-deficient. In a further embodiment, the medium is a serum-free medium.

A suitable medium is as follows:

| Working pH range | 7.2-7.4 | |
|---|---|---|
| Components | Mg/L (unless otherwise indicated) | |
| IGF-II | 1-750 | nM |
| Plasminogen | 0.01-50 | μg/ml |

Inorganic Salts

| | |
|---|---|
| CaCl₂—2H₂O | 250.00 |
| KCL | 186.38 |
| KH₂PO₄ | 47.99 |
| MgSO₄ | — |
| MgSO₄7H₂O | 49.30 |
| NaCl | 5551.80 |
| NaHCO₃ | 2100.25 |

Other Components

| | |
|---|---|
| EDTA | 3.72 |
| D-Glucose | 36.03 |
| Sodium Lactate | 1121.00 |
| Lactate NaSalt (ml/L) 1.42 | — |
| Sodium Pyruvate | 22.00 |
| HSA | 1000.00 |
| Phenol Red | — |

Amino Acids

| | |
|---|---|
| L-Arginine | 63.20 |
| L-Cystine | 12.02 |
| L-Cystine-2HCL | — |
| L-Glutamine | 146.15 |
| Glycine | 3.75 |
| L-Histidine | — |
| L-Histidine•HCl•H₂O | 20.96 |
| L-Isoleucine | 26.23 |
| L-Leucine | 26.24 |
| L-Lysine | — |
| L-Lysine•HCl | 36.52 |
| L-Methionine | 7.46 |
| L-Phenylalanine | 16.52 |
| L-Serine | 5.26 |
| L-Threonine | 23.82 |
| L-Tryptophan | 5.11 |
| L-Tyrosine | 18.12 |
| L-Tyrosine NaH₂O | — |
| L-Valine | 23.42 |
| L-Alanine | 4.45 |
| L-Asparagine | — |
| L-Asparagine-H₂O | 7.50 |
| L-Aspartic Acid | 6.66 |
| L-Glutamic Acid | 7.36 |
| L-Proline | 5.76 |

Antibiotics

| | |
|---|---|
| Pen G Na Salt (units) | 100,000.00 |
| Strep Sulfate | 50.00 |

The present invention is also anticipated to be suitable for promoting implantation of an isolated embryo into uterine endometrium.

Accordingly, in another embodiment the present invention provides a method of promoting implantation of an isolated embryo into uterine endometrium, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The isolated embryo may be an embryo in vitro, or alternatively, an isolated embryo that has been introduced into a subject. In this regard, it will be understood that the present invention not only contemplates the exposure of an isolated embryo in vitro to IGF-II and either or both of plasminogen and uPA prior to and/or concurrently with implantation, but also an isolated embryo introduced into a subject and subsequently exposed to IGF-II and either or both of plasminogen and uPA in vivo.

In this regard, introduction of an isolated embryo into a subject and subsequent exposure to IGF-II, and either or both of plasminogen and uPA is particularly suitable for females diagnosed to be at risk of miscarriage, pre-eclampsia, intrauterine growth restriction, pre-term birth or placental abruption. Methods for assessing the risk to such events or conditions are known in the art.

The present invention also provides an embryo with improved implantation capacity produced by this method. The present invention also provides a non-human animal produced from such an embryo.

The method of this embodiment of the present invention is also anticipated to be suitable for promoting the implantation into uterine endometrium of an embryo produced from an isolated oocyte, by exposure of the isolated oocyte to IGF-II and either or both of plasminogen and uPA.

Accordingly, in another embodiment the present invention provides a method of promoting implantation into uterine endometrium of an embryo produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides an oocyte with improved implantation capacity produced by this method. The present invention also provides a non-human animal produced from such an embryo or oocyte.

The isolated oocyte may be an oocyte in vitro, or alternatively, an isolated oocyte present that has been introduced into a subject. In this regard, it will be understood that the present invention not only contemplates the exposure of an isolated oocyte in vitro to IGF-II and either or both of plasminogen and uPA prior to, and/or concurrently with, fertilization and/or implantation, but also an isolated oocyte introduced into a subject and subsequently exposed to IGF-II and either or both of plasminogen and uPA.

Introduction of an isolated oocyte into a subject and subsequent exposure to IGF-II, and either or both of plasminogen and uPA is also suitable for females diagnosed to be at risk of miscarriage, pre-eclampsia, intrauterine growth restriction, pre-term birth or placental abruption.

In the case of exposing embryos and oocytes in vitro, it is anticipated that a medium including IGF-II and either or both of plasminogen and urokinase plasminogen activator may be used to culture an embryo and/or oocyte to improve implantation.

Accordingly, in another embodiment the present invention provides an oocyte and/or embryo culture medium for promoting implantation of an embryo into uterine endometrium, the culture medium including 0.0003 to 750 ng/ml IGF-II, or a variant or analogue thereof, and further including either or both of 0.01 to 50 μg/ml plasminogen,

The present invention also provides a combination product for promoting implantation of an embryo into uterine endometrium. A combination product including culture medium, IGF-II, and either or both of plasminogen and uPA is as previously discussed.

The present invention is also anticipated to be suitable for the production of compositions for exposure to isolated embryos and/or isolated oocytes to improve implantation.

Accordingly, in another embodiment the present invention provides a composition for exposure to an isolated embryo to promote implantation of the isolated embryo into uterine endometrium, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

In another embodiment, the present invention provides a composition for exposure to an isolated oocyte to promote implantation into uterine endometrium of an embryo produced from the isolated oocyte, the composition including 0.0003 to 750 ng/ml IGF-11, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

A suitable composition is the medium as previously discussed.

The present invention is also anticipated to be suitable for reducing the likelihood of failure of an isolated embryo to implant into uterine endometrium.

Accordingly, in another embodiment the present invention provides a method of reducing the likelihood of failure of an isolated embryo to implant into uterine endometrium, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The isolated embryo may be an embryo in vitro, or alternatively, an isolated embryo that has been introduced into a subject. In this regard, it will be understood that the present invention not only contemplates the exposure of an isolated embryo in vitro to IGF-II and either or both of plasminogen and uPA prior to, and/or concurrently with, implantation, but also an isolated embryo introduced into a subject and subsequently exposed to IGF-II and either or both of plasminogen and uPA in vivo.

In this regard, introduction of an isolated embryo into a subject and subsequent exposure to IGF-II, and either or both of plasminogen and uPA is also anticipated to be suitable for females diagnosed to be at risk of miscarriage, pre-eclampsia, intrauterine growth restriction, pre-term birth or placental abruption.

The present invention also provides an embryo with a reduced likelihood of failure produced by this method. The present invention also provides a non-human animal produced from such an embryo.

The method of the present invention is also anticipated to be suitable for reducing the likelihood of implantation failure of an embryo produced from an isolated oocyte, by exposure of the isolated oocyte to IGF-II and either or both of plasminogen and uPA.

Accordingly, in another embodiment the present invention provides a method of reducing the likelihood of implantation failure into uterine endometrium of an embryo produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides an oocyte with reduced likelihood of implantation failure produced by this method. The present invention also provides an embryo or a non-human animal produced from the oocyte.

The isolated oocyte may be an oocyte in vitro, or alternatively, an isolated oocyte present that has been introduced into a subject. In this regard, it will be understood that the present invention not only contemplates the exposure of an isolated oocyte in vitro to IGF-II and either or both of plasminogen and uPA prior to, and/or concurrently with, fertilization and/or implantation, but also an isolated oocyte introduced into a subject and subsequently exposed to IGF-II and either or both of plasminogen and uPA.

Introduction of an isolated oocyte into a subject and subsequent exposure to IGF-II, and either or both of plasminogen and uPA is also anticipated to be suitable for females diagnosed to be at risk of miscarriage, pre-eclampsia, intrauterine growth restriction, pre-term birth or placental abruption.

In the case of exposing embryos and oocytes in vitro, it is anticipated that a medium including IGF-II and either or both of plasminogen and uPA may be used to culture an embryo and/or oocyte to improve implantation.

Accordingly, in another embodiment the present invention provides an oocyte and/or embryo culture medium for reducing the likelihood of failure of an embryo to implant into uterine endometrium, the culture medium including 0.0003 to 750 ng/ml IGF-II, or a variant or analogue thereof, the medium further including either or both of 0.01 to 50 μg/ml plasminogen, or a variant or analogue thereof, and 0.01 to 50 μg/ml urokinase plasminogen activator, or a variant or analogue thereof.

The present invention also provides a combination product for reducing the likelihood of failure of an embryo to implant into uterine endometrium. A combination product including culture medium, IGF-II, and either or both of plasminogen and uPA is as previously discussed.

The present invention is also anticipated to be suitable for the production of compositions for exposure to isolated embryos and/or isolated oocytes to reduce the likelihood of implantation failure.

Accordingly, in another embodiment the present invention provides a composition for exposure to an isolated embryo to reduce the likelihood of failure of the isolated embryo to implant into uterine endometrium, the composition including including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

In another embodiment, the present invention provides a composition for exposure to an isolated oocyte to reduce the likelihood of failure of an embryo produced from the isolated oocyte to implant into uterine endometrium, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

A suitable composition is the medium as previously discussed.

The present invention is also anticipated to be suitable for improving development of a placenta formed from an embryo exposed to IGF-II and either or both of plasminogen and uPA following implantation of the embryo into uterine endometrium.

Thus, the present invention is also anticipated to be suitable for the production of a culture medium for embryos and oocytes to improve placental development.

Accordingly, in another embodiment the present invention also provides an oocyte and/or embryo culture medium for improving placental development, the medium including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for improving placental development of embryos.

Accordingly, in another embodiment the present invention provides a method of improving placental development for an isolated embryo implanted into uterine endometrium, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides an embryo with improved placental development capacity produced by this method. The present invention also provides a non-human animal produced from such an embryo.

Methods for determining the extent of placental development are known in the art. For example, ultrasonography can be used to measure the size and dimensions of the placenta and its density.

The present invention is also anticipated to be suitable for improving placental development of an embryo produced from an isolated oocyte.

Accordingly, in another embodiment the present invention provides a method of improving placental development for an embryo implanted into uterine endometrium, the embryo being produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides an oocyte with improved placental development capacity produced by this method. The present invention also provides an embryo and a non-human animal produced from such an oocyte.

The present invention also contemplates compositions for exposure to embryos and/or oocytes that are anticipated to improve placental development.

Accordingly, in another embodiment the present invention provides a composition for exposure to an isolated embryo to improve development of a placenta formed by implantation of the isolated embryo into uterine endometrium, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

In another embodiment, the present invention also provides a composition for exposure to an isolated oocyte to improve development of a placenta formed by implantation into uterine endometrium of an embryo produced from the isolated oocyte, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for the production of a culture medium for an embryo and/or oocyte to improve development of a placenta formed by implantation of an embryo.

The present invention also provides a combination product for improving development of a placenta formed by implantation of an embryo into uterine endometrium. A combination product including culture: medium, IGF-II, and either or both of plasminogen and uPA is as previously discussed.

The present invention is also anticipated to be suitable for improving placental growth and function. Thus the present invention contemplates culture medium for an embryo and/or oocyte to improve placental growth and/or function, and methods and compositions for improving placental growth and function.

Accordingly, in another embodiment the present invention provides an oocyte and/or embryo culture medium for improving placental growth and/or function, the medium including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for improving placental growth and/or function for an embryo.

Accordingly, in another embodiment the present invention provides a method of improving placental growth and/or function for an isolated embryo implanted into uterine endometrium, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

The present invention also provides an embryo produced by this method. The present invention also provides a non-human animal produced from such an embryo.

Methods for determining the extent of placental growth and/or function are known in the art.

The present invention is also anticipated to be suitable for improving placental growth and/or function of an embryo produced from an isolated oocyte.

Accordingly, in another embodiment the present invention provides a method of improving placental growth and/or function for an embryo implanted into uterine endometrium, the embryo being produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides an oocyte produced by this method. The present invention also provides an embryo and a non-human animal produced from such an oocyte.

The present invention also contemplates compositions for exposure to embryos and/or oocytes that are anticipated to improve placental growth and/or function.

Accordingly, in another embodiment the present invention provides a composition for exposure to an isolated embryo to improve growth and/or function of a placenta formed by implantation of the isolated embryo into uterine endometrium, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

In another embodiment, the present invention provides a composition for exposure to an isolated oocyte to improve growth and/or function of a placenta formed by implantation into uterine endometrium of an embryo produced from the isolated oocyte, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention also provides a combination product for improving growth and/or function of a placenta formed by implantation of an embryo into uterine endometrium.

A combination product including culture medium, IGF-II, and either or both of plasminogen and uPA as previously discussed.

The present invention is also anticipated to be suitable for reducing the extent and/or likelihood of miscarriage occurring in a subject with an isolated embryo introduced into the subject, or an embryo produced from an isolated oocyte introduced into the subject.

Accordingly, in another embodiment the present invention provides a method of reducing the likelihood of miscarriage in a subject with an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

A reduction in the likelihood of miscarriage is a decreased probability that miscarriage may occur in a subject, as compared to the probability that the same event may occur in another subject with similar risk factors.

In one embodiment, the subject in the various embodiments of the present invention is a human subject.

In the case of this embodiment of the present invention, the subject may be for example a human subject susceptible to miscarriage, and in one form, susceptible to recurrent spontaneous miscarriage.

In a further embodiment, the present invention provides a method of reducing the likelihood of miscarriage in a subject with an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The miscarriage occurring in the subject in the relevant various embodiments of the present invention may be miscarriage due to recurrent spontaneous miscarriage, otherwise known as recurrent spontaneous abortion.

The present invention also provides compositions for exposure to isolated embryo and isolated oocytes that are anticipated to reduce the extent and/or likelihood of miscarriage.

Accordingly, in another embodiment the present invention provides a composition for exposure to an isolated embryo to reduce the likelihood of miscarriage in a subject with an isolated embryo introduced into the subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

In a further embodiment, the present invention provides a composition for exposure to an isolated oocyte to reduce the likelihood of miscarriage in a subject with an embryo produced from the isolated oocyte introduced into the subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for the production of a culture medium for an embryo and/or oocyte to reduce the likelihood of miscarriage in a subject into which the embryo or oocyte is introduced.

The present invention also provides a combination product for exposure to an isolated embryo and/or oocyte to reduce the likelihood of miscarriage developing in a subject into which the oocyte or embryo is introduced. A combination product including culture medium, IGF-II, and either or both of plasminogen and uPA is as previously discussed.

The present invention is also anticipated to be suitable for reducing the extent and/or likelihood of pre-eclampsia occurring in a subject with an isolated embryo introduced into the subject, or an embryo produced from an isolated oocyte introduced into the subject.

Accordingly, in another embodiment the present invention provides a method of reducing the extent and/or likelihood of pre-eclampsia in a subject with an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

A reduction in the likelihood of pre-eclampsia is a decreased probability that pre-eclampsia may occur in a subject, as compared to the probability that the same event may occur in another subject with similar risk factors.

A reduction in the extent of abruption in a subject is a reduction and/or amelioration in the degree of pre-eclampsia that may occur in a particular subject susceptible to, or actually suffering from, pre-eclampsia.

In one embodiment, the subject is a human subject susceptible to developing pre-eclampsia.

In a further embodiment, the present invention provides a method of reducing the extent and/or likelihood of pre-eclampsia in a subject with an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides compositions for exposure to isolated embryos and isolated oocytes that are anticipated to reduce the extent and/or likelihood of pre-eclampsia.

Accordingly, in another embodiment the present invention provides a composition for exposure to an isolated embryo to reduce the extent and/or likelihood of pre-eclampsia in a subject with the isolated embryo introduced into the subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

A suitable composition is the media described previously.

In a further embodiment, the present invention provides a composition for exposure to an isolated oocyte to reduce the extent and/or likelihood of pre-eclampsia in a subject with an embryo produced from the isolated oocyte introduced into the subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for the production of a culture medium for an embryo and/or oocyte to reduce the extent and/or likelihood of pre-eclampsia in a subject into which the embryo or oocyte is introduced.

The present invention also provides a combination product for exposure to an isolated embryo and/or oocyte for reducing the extent and/or likelihood of pre-eclampsia in a subject into which the oocyte or embryo is introduced. A combination product including culture medium, IGF-II, and either or both of plasminogen and uPA are as previously discussed.

The present invention is also anticipated to be suitable for reducing the extent and/or likelihood of intrauterine growth restriction (fetal growth restriction) occurring in a subject with an isolated embryo introduced into the subject, or an embryo produced from an isolated oocyte introduced into the subject.

Accordingly, in another embodiment the present invention provides a method of reducing the extent and/or likelihood of intrauterine growth restriction in a subject with an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

In one embodiment, the subject is a human subject susceptible to intrauterine growth restriction.

A reduction in the likelihood of intrauterine growth restriction is a decreased probability that such an event may occur in a subject, as compared to the probability that the same event may occur in another subject with similar risk factors.

A reduction in the extent of intrauterine growth restriction in a subject is a reduction and/or amelioration in the degree of intrauterine growth restriction that may occur in a particular subject susceptible to intrauterine growth restriction.

In a further embodiment, the present invention provides a method of reducing the extent and/or likelihood of intrauterine growth restriction in a subject with an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides compositions for exposure to isolated embryos and isolated oocytes that are anticipated to reduce the extent and/or likelihood of intrauterine growth restriction.

Accordingly, in another embodiment the present invention provides a composition for exposure to an isolated embryo to reduce the extent and/or likelihood of intrauterine growth restriction in a subject with the isolated embryo introduced into the subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

In a further embodiment, the present invention provides a composition for exposure to an isolated oocyte to reduce the extent and/or likelihood of intrauterine growth restriction in a subject with an embryo produced from the isolated oocyte introduced into the subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for the production of a culture medium for an embryo and/or oocyte to reduce the extent and/or likelihood of intrauterine growth restriction in a subject into which the embryo or oocyte is introduced.

The present invention also provides a combination product for exposure to an isolated embryo and/or oocyte to reduce the extent and/or likelihood of intrauterine growth restriction in a subject into which the embryo or oocyte is introduced. A combination product including culture medium, IGF-II, and either or both of plasminogen and uPA is as previously discussed.

The present invention is also anticipated to be suitable for reducing the likelihood of pre-term labour and reducing the likelihood of pre-term delivery of a fetus occurring in a subject with an isolated embryo introduced into the subject, or an embryo produced from an isolated oocyte introduced into the subject.

Accordingly, in another embodiment the present invention provides a method of reducing the likelihood of pre-term delivery of a fetus produced from an isolated embryo introduced into a subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

In one embodiment, the subject is a human subject susceptible to pre-term labour and delivery.

A reduction in the likelihood of pre-term labour and delivery is a decreased probability that such an event may occur in a subject, as compared to the probability that the same event may occur in another subject with similar risk factors.

In a further embodiment, the present invention provides a method of reducing the likelihood of pre-term delivery of a fetus resulting from an embryo produced from an isolated oocyte introduced into a subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides compositions for exposure to isolated embryos and isolated oocytes that are anticipated to reduce the likelihood of pre-term delivery of a fetus.

Accordingly, in another embodiment the present invention provides a composition for exposure to an isolated embryo to reduce the likelihood of pre-term delivery of a fetus produced from the isolated embryo introduced into a subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

In a further embodiment, the present invention provides a composition for exposure to an isolated oocyte to reduce the likelihood of pre-term delivery of a fetus produced from the isolated oocyte introduced into a subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for the production of a culture medium for an embryo and/or oocyte to reduce the likelihood of pre-term delivery of a fetus produced from the isolated embryo or oocyte introduced into a subject.

The present invention also provides a combination product for exposure to an isolated embryo and/or oocyte to reduce the likelihood of pre-term delivery of a fetus produced from the isolated oocyte or embryo introduced into a subject. A combination product including culture medium, IGF-II, and either or both of plasminogen and uPA is as previously discussed.

The present invention is also anticipated, to be suitable for increasing the likelihood that a fetus is carried to term or near term in a subject with an isolated embryo introduced into the subject, or an embryo produced from an isolated oocyte introduced into the subject.

Accordingly, in another embodiment the present invention provides a method of increasing the likelihood that a fetus is carried to term or near term in a subject, the fetus produced from an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

An increase in the likelihood that a fetus is carried to term or near term is an increased probability that such an event may occur in a subject, as compared to the probability that the same event may occur in another subject with similar risk factors. In one embodiment, the subject is susceptible to pre-term delivery of a fetus.

In a further embodiment, the present invention provides a method of increasing the likelihood that a fetus is carried to term or near term in a subject, the fetus produced from an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

In this regard, the carrying of a fetus to term is understood to mean carrying the fetus to 37 to 40 weeks of gestation.

The present invention also provides compositions for exposure to isolated embryos and isolated oocytes that are anticipated to increase the likelihood that a fetus resulting from introduction of the isolated embryo or an isolated oocyte into a subject is carried to term or near term.

Accordingly, in another embodiment the present invention provides a composition for exposure to an isolated embryo to increase the likelihood that a fetus resulting from introduction of the isolated embryo into a subject is carried to term or near term, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

In a further embodiment, the present invention provides a composition for exposure to an isolated oocyte to increase the likelihood that a fetus resulting from introduction into the subject of an embryo produced from the isolated oocyte is carried to term or near term, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for the production of a culture medium for an embryo and/or oocyte to increase the likelihood that a fetus produced from the embryo or oocyte introduced into a subject is carried to term or near term.

The present invention also provides a combination product for exposure to an isolated embryo and/or oocyte to increase the likelihood that a fetus resulting from introduction into a subject of an embryo or oocyte is carried to term or near term. A combination product including culture medium, IGF-II, and either or both of plasminogen and uPA is as previously discussed.

The present invention is also anticipated to be suitable for normalising the length of the gestation period of a fetus carried by a subject with an isolated embryo introduced into the subject, or an embryo produced from an isolated oocyte introduced into the subject.

Accordingly, in another embodiment the present invention provides a method of normalising the length of the gestation period of a fetus carried by a subject, the fetus being produced from an isolated embryo introduced into the subject, the method including the step of exposing the isolated embryo to 0.0003 to 750 ng/ml IGF-II, or a variant or analogue thereof, the method further including the step of exposing the embryo to either or both of 0.01 to 50 μg/ml plasminogen, or a variant or analogue thereof, and 0.01 to 50 μg/ml urokinase plasminogen activator, or a variant or analogue thereof.

In a further embodiment, the present invention provides a method of normalising the length of the gestation period of a fetus carried by a subject, the fetus being produced from an embryo produced from an isolated oocyte introduced into the subject, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides compositions for exposure to isolated embryos and isolated oocytes that are anticipated to normalise the length of the gestation period of a fetus resulting from isolated embryos and isolated oocytes introduced into the subject.

Accordingly, in another embodiment the present invention provides a composition for exposure to an isolated embryo to normalise the length of the gestation period of a fetus resulting from the isolated embryo introduced into a subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

In a further embodiment, the present invention provides a composition for exposure to an isolated oocyte to normalise the length of the gestation period of a fetus resulting from the isolated oocyte introduced into a subject, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for the production of a culture medium for an embryo and/or oocyte to normalise the length of the gestation period of a fetus resulting from introduction of the oocyte or embryo into a subject.

The present invention also provides a combination product for exposure to an isolated embryo and/or oocyte to normalise the length of the gestation period of a fetus resulting from introduction of the oocyte or embryo into a subject. A combination product including culture medium, IGF-II, and either or both of plasminogen and uPA is as previously discussed.

The present invention is also anticipated to be suitable for reducing the extent and/or likelihood of placental abruption occurring in a subject with an isolated embryo introduced into the subject, or an embryo produced from an isolated oocyte introduced into the subject.

Accordingly, in another embodiment the present invention provides a method of reducing the extent and/or likelihood of placental abruption in a subject, the method including the step of exposing an isolated embryo or an isolated oocyte for introduction into the subject to 0.0003 to 750 ng/ml IGF-II, or a variant or analogue thereof, the method further including the step of exposing the oocyte or embryo to either or both of 0.01 to 50 μg/ml plasminogen, or a variant or analogue thereof, and 0.01 to 50 μg/ml urokinase plasminogen activator, or a variant or analogue thereof.

In the case of the present embodiment of the invention, the subject may also be a subject that is susceptible to placental abruption.

A reduction in the likelihood of placental abruption in a subject is a decreased probability that placental abruption may occur in a subject, as compared to the probability that the same event may occur in another subject with similar risk factors.

A reduction in the extent of placental abruption in a subject is a reduction and/or amelioration in the amount of placental abruption that may occur in a particular subject susceptible to, or actually undergoing, placental abruption.

The present invention is also anticipated to be suitable for reducing the extent and/or likelihood of placental abruption occurring in a subject with an embryo produced from an isolated oocyte introduced into the subject.

Accordingly, in another embodiment the present invention provides a method of reducing the extent and/or likelihood of abruption of a placenta formed by implantation into uterine endometrium of an embryo produced from an isolated oocyte, the method including the step of exposing the isolated oocyte to 0.0003 to 750 ng/ml IGF-II, or a variant or analogue thereof, the method further including the step of exposing the oocyte to either or both of 0.01 to 50 μg/ml plasminogen, or a variant or analogue thereof, and 0.01 to 50 μg/ml urokinase plasminogen activator, or a variant or analogue thereof.

The present invention also provides an oocyte produced according to this method.

In the case of an oocyte and/or embryo in vitro, it is anticipated that the present invention may be used to produce a culture medium for an embryo and/or oocyte to reduce the extent and/or likelihood of placental abruption that occurs in a subject with an isolated embryo introduced into the subject, or for reducing the extent and/or likelihood of placental abruption that occurs in a subject with an isolated oocyte introduced into the subject.

The present invention also provides compositions for exposure to isolated embryos and isolated oocytes that are anticipated to reducing the extent and/or likelihood of placental abruption.

Accordingly, in another embodiment the present invention provides a composition for exposure to an isolated embryo to reduce the extent and/or likelihood of abruption of a placenta formed by implantation of the isolated embryo into uterine endometrium, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

A suitable composition is the medium as previously described.

In a further embodiment, the present invention also provides a composition for exposure to an isolated oocyte to reduce the extent and/or likelihood of abruption of a placenta formed by implantation into uterine endometrium of an embryo produced from the isolated oocyte, the composition including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention is also anticipated to be suitable for the production of a culture medium for an embryo and/or oocyte to reduce the extent and/or likelihood of abruption of a placenta formed by implantation of the isolated embryo into uterine endometrium.

Accordingly, in another embodiment the present invention provides an oocyte and/or embryo culture medium for reducing the extent and/or likelihood of abruption of a placenta formed by implantation of the isolated embryo into uterine endometrium, the medium including 0.0003 to 750 ng/ml IGF-II, or a variant or analogue thereof, and further including either or both of 0.01 to 50 μg/ml plasminogen, or a variant or analogue thereof, and 0.01 to 50 μg/ml urokinase plasminogen activator, or a variant or analogue thereof.

The present invention also provides a combination product for exposure to an isolated embryo and/or oocyte for reducing the extent and/or likelihood of abruption of a placenta formed by implantation of the isolated embryo into uterine endometrium. A combination product including culture medium, IGF-II, and either or both of plasminogen and uPA are as previously discussed.

The present invention also provides methods for improving post-natal outcome due to improved placental development and/or a reduction in the risk and/or likelihood of pregnancy complications due to deficient placental development. The present invention also provides compositions, media and combination products to improve post-natal outcomes.

In the case of an embryo or oocyte in vitro, the exposure of an oocyte or embryo in the various embodiments of the present invention to IGF-II, and either or both of plasminogen and urokinase plasminogen activator will as previously discussed herein generally occur in a suitable medium, and may include one or more further agents as required. Suitable media are as previously described.

It will be appreciated, however, that the exposure of the embryo or oocyte in vitro to IGF-II, and either or both of plasminogen and uPA, may be exposure to these agents at the same time, or alternatively, be separate exposure to one or more of these agents.

It will also be appreciated that depending upon the timing of the exposure of the embryo or oocyte (in vitro or in vivo) to IGF-II, plasminogen, and uPA, it may also be possible to improve implantation without a subsequent effect on placental development, and conversely, it may be possible to improve placental development without an improvement in implantation.

In the case of in vivo exposure of isolated embryos and oocytes, the IGF-II, plasminogen and uPA may be administered separately or together, in the form of suitable pharmaceutical compositions.

The administration of IGF-II, and either or both of plasminogen and uPA as a pharmaceutical composition in the various relevant embodiments of the present invention may be within any time suitable to produce the desired effect. IGF-II, plasminogen and uPA may be administered orally, parenterally, topically or by any other suitable means, and therefore transit time must be taken into account.

The administration of IGF-II, plasminogen and uPA in the various relevant embodiments of the present invention may also include the use of one or more pharmaceutically acceptable additives, including pharmaceutically acceptable salts, amino acids, polypeptides, polymers, solvents, buffers, excipients and bulking agents, taking into consideration the particular physical and chemical characteristics of the IGF-II, plasminogen and uPA administered.

For example, IGF-II, plasminogen and uPA can be prepared into a variety of pharmaceutical compositions in the form of, e.g., an aqueous solution, an oily preparation, a fatty emulsion, an emulsion, a gel, etc., and these preparations can be administered as intramuscular or subcutaneous injection or as injection to an organ (including the heart), or as an embedded preparation or as a transmucosal preparation through nasal cavity, rectum, uterus, vagina, uterus, lung, etc. The composition may be administered in the form of oral preparations (for example solid preparations such as tablets, capsules, granules or powders; liquid preparations such as syrup, emulsions or suspensions). Compositions containing the agent may also contain a preservative, stabiliser, dispersing agent, pH controller or isotonic agent. Examples of suitable preservatives are glycerin, propylene glycol, phenol or benzyl alcohol. Examples of suitable stabilisers are dextran, gelatin, α-tocopherol acetate or alpha-thioglycerin. Examples of suitable dispersing agents include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include acetic acid. Examples of suitable isotonic agents are glucose, D-sorbitol or D-mannitol.

The administration of IGF-II, plasminogen and uPA in the various relevant embodiments of the present invention may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, flavorant or sweetener, taking into account the physical and chemical properties of IGF-II, plasminogen and uPA being administered.

For these purposes, the composition may be administered orally, parenterally, by inhalation spray, adsorption, absorption, topically, rectally, nasally, bucally, vaginally, via the uterus, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, intrasternal, and intracranial injection or infusion techniques.

When administered parenterally, the composition will normally be in a unit dosage, sterile injectable form (solution, suspension or emulsion) which is usually isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents, for example, as solutions in 1,3-butanediol. Among the acceptable vehicles and solvents that may be employed are water, saline, Ringer's solution, dextrose solution, isotonic sodium chloride solution, acetic acid and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain minor amounts of additives, such as substances that enhance solubility, isotonicity, and chemical stability, for example anti-oxidants, buffers and preservatives.

When administered orally, the IGF-II, plasminogen and uPA will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

A tablet may be made by compressing or molding IGF-II, plasminogen and uPA with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Molded tablets may be made by molding in a suitable machine, a mixture of the powdered active ingredient and a suitable carrier moistened with an inert liquid diluent.

The administration of IGF-II, plasminogen and uPA in the various relevant embodiments of the present invention may also utilize controlled release technology. IGF-II, plasminogen and uPA may also be administered as a sustained-release pharmaceutical. To further increase the sustained release effect, IGF-II, plasminogen and uPA may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, IGF-II, plasminogen and uPA may be incorporated into a hydrophobic polymer matrix for controlled release over a period of days. IGF-II and either or both of plasminogen and uPA may then be molded into a solid implant, slow release mesh or externally applied patch, suitable for providing efficacious concentrations of the agent over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time release characteristics and release kinetics. IGF-II, plasminogen and uPA may then be molded into a solid implant suitable for providing efficacious concentrations of IGF-II, plasminogen and uPA over a prolonged period of time without the need for frequent re-dosing. IGF-II, plasminogen and uPA can be incorporated into the biodegradable polymer or polymer mixture in any suitable manner known to one of ordinary skill in the art and may form a homogeneous matrix with the biodegradable polymer, or may be encapsulated in some way within the polymer, or may be molded into a solid implant.

Methods for preparing various dosage forms are known in the art, for example as described in Remington's Pharmaceutical Sciences, 18th Ed. (Easton, Pa.: Mack Publishing Company, 1990).

Suitable amounts for delivery are administration of 1 mg/kg/day of IGF-II, 0.01 mg/kg/day plasminogen and 0.01 mg/kg/day uPA.

In one embodiment, IGF-II, plasminogen and uPA are delivered via a vaginal route or via the uterus. Alternatively, IGF-II may be delivered subcutaneously, and either or both of plasminogen and uPA delivered via a vaginal route or via the uterus.

In this regard, it has been recognised that implantation of an embryo into uterine endometrium may be promoted by intravaginally exposing a female subject to IGF-II, and either or both of plasminogen and uPA.

Accordingly, in another embodiment the present invention provides an intravaginal composition, the composition including IGF-II, or a variant or an analogue thereof; and either or both of plasminogen, or a variant or an analogue thereof, and urokinase plasminogen activator, or a variant or an analogue thereof.

Methods are known in the art for intravaginally exposing female subjects to one or more agents, including the use of gels, ointments and pessaries to deliver the agents.

In one embodiment, the concentration of IGF-II in the intravaginal composition is such to provide a concentration of IGF-II in the reproductive organs in the range from 1 to 750 ng IGF-II/100 mg tissue.

In one embodiment, the concentration of plasminogen in the composition is such to provide a concentration of plasminogen in the reproductive organs in the range from 0.01 to 50 μg/100 mg tissue.

In one embodiment, the concentration of urokinase plasminogen activator in the composition is such to provide a concentration in the reproductive organs in the range from 0.01 to 50 μg/100 mg tissue.

In this regard, delivery of IGF-II, and either or both of plasminogen and uPA by a transvaginal delivery system is anticipated to lead to a significantly increased concentration and efficacy of these agents to the reproductive organs, as compared to other routes of administration, such as oral administration.

The delivery route utilizes the composition directly or incorporated into an intravaginal device for transmucosal delivery of IGF-II, and for delivery of either or both of plasminogen and uPA with for example a mucoadhesive agent, one or more carriers and, optionally, permeation enhancing agents and solubilizing excipients for transvaginal delivery.

It will be appreciated that IGF-II, and either or both of plasminogen and uPA may be administered alone or in combination with other pharmacological agents or a pharmaceutically acceptable excipient.

The composition may be delivered by a suitable method, including administering the composition directly to the vagina as a solution, gel, cream, lotion, ointment, foam, film, suppository, liposomal suspension, microemulsion, capsule, tablet, microparticles, microcapsules, nanoparticles, nanocapsules, or by inserting an intravaginal device for delivery of the agents. Devices suitable for these purposes include an intravaginal tampon, intravaginal ring, intravaginal pessary, intravaginal sponge, intravaginal tablet, intravaginal capsule, intravaginal patch, intravaginal iontophoretic system, intravaginal cup, and intravaginal strip.

A composition of the invention for transmucosal delivery typically includes a number of additional components in addition to IGF-II, and either or both of plasminogen and uPA, such as a mucoadhesive agent that provides close contact of the composition with the vaginal epithelium, a lipophilic or hydrophilic carrier that assures safe patient handling and enhances surface exposure of the drug to the vaginal mucosa, and a permeation enhancer that facilitates transfer of the pharmacologically active agents across the epithelial barriers.

For topical delivery to the vaginal mucosa, the composition typically includes two components in addition to IGF-II, and either or both of plasminogen and uPA, namely a mucoadhesive agent, and a lipophilic or hydrophilic carrier. IGF-II and either or both of plasminogen and uPA are present in the composition at a dose sufficient to assert its therapeutic effect, typically from about 0.001 to about 100 mg, for example from 0.1 to 50 mg or from 1 to 20 mg.

The composition is typically formulated in therapeutic unit dosage forms including the active agents alone or in combination with other pharmacological agents or pharmaceutically acceptable excipients for intravaginal or transvaginal delivery to a female subject.

The composition typically contains from 0.1 to about 100 mg, for example from 1 to 20 mg, of IGF-II and 0.001 to about 40 mg, for example from 0.1 to 10 mg plasminogen, and a similar amount of uPA, from about 0.1 to about 25% of mucoadhesive agent promoting adhesion of the composition to the vaginal mucosa, from about 5 to about 30% of a permeation enhancer assuring transfer of the drug across the vaginal epithelium, and from about 40 to about 95% of a lipophilic or hydrophilic carrier serving as a vehicle for the drug and, optionally, from about 0 to about 30%, and generally about 1 to 5%, of a solubilizing agent.

In one embodiment, the intravaginal delivery device will be replaced once a day.

Other pharmaceutically acceptable excipients suitable for vaginal delivery, such as buffers, fillers, stabilizers, emulsifiers, and any such other excipient as is known in the art to be useful for these purposes may also be added.

The composition may be formulated as a solution, gel, cream, lotion, ointment, foam, film, suppository, liposomal suspension, microemulsion, capsule, tablet, microparticles, microcapsules, nanoparticles, or nanocapsules, and can be delivered as stand alone or incorporated within an intravaginal device.

As discussed previously, the composition formulated as above can be incorporated into an intravaginal device or used as a coating for such a device, for example, a tampon or tampon-like device medicated or coated with the above described mucoadhesive composition. Alternatively, the composition may be incorporated into a sponge, foam, film, tablet, capsule, ring, mucoadhesive patch, iontophoretic system, strip, pessary, or other material. Absorbent material or matrix of such a device may be impregnated with a active agent-containing solution, suspension, lotion, cream, emulsion, microemulsion, liposomes, microparticles, microcapsules, nanoparticles, or nanocapsules.

In one embodiment, the mucoadhesive agent is a polymeric compound, such as a cellulose derivative but it may also be a natural gum, alginate, pectin, or such similar polymer. The mucoadhesive agent is typically present in from about 0.1 to about 25%, by weight, usually in from about 1.5 to about 15%, such as about 1.5-5%.

Sorption promoters are generally present from about 2 to about 30%, by weight. Sorption promoters include non-ionizable glycol ester derivatives, such as polyethylene glycol caprylic/capric glycerides, glycol derivatives with glycerol esters, such as oleic acid esters of propylene glycol and glycerol. A suitable non-ionizable glycol ether derivatives, such asethoxydiglycol.

The composition may additionally include a lipophilic or hydrophilic carrier that is appropriate for use with IGF-II and either or both of plasminogen anduPA. Such carrier is typically present from about 30 to about 95%, by weight. Preferred lipophilic carriers include any medium chain triglycerides and/or a saturated mono-, di- or triglyceride of fatty acids, particularly those having carbon chain of from 8 to 18 carbons, or a mixture thereof.

Such hydrophilic carriers include polyethylene glycols of molecular weight between about 200 and 8000, or derivatives or mixtures thereof, such as PEG 6000/PEG 1500, or PEG 6000/PEG 1500/PEG 400, or PEG 6000/PEG 400, or PEG 8000/PEG 1500.

The composition may additionally contain penetration enhancers, compounds which assist in improving penetration properties of the drug or their mixtures by changing the surface properties of the active agents. Examples of penetration enhancers are non-ionic surfactants.

The composition may also include a solubilizing agent, such as acetic acid, complex-forming solubilizer citric acid, ethylenediamine-tetraacetate, sodium meta-phosphate, succinic acid, urea, cyclodextrin, polyvinylpyrrolidone, diethylammonium-ortho-benzoate, or micell-forming solubilizers such as tweens and spans, for example Tween 80. Other solubilizer useful for the compositions of this invention are polyoxyethylene sorbitan fatty acid ester, polyoxyethylene n-alkyl ethers, n-alkyl amine n-oxides, poloxamers, organic solvents, phospholipids and cyclodextrines. The solubilizing agents may be added from about 0.1% to about 30%.

The composition may additionally contain other excipients, such as, fillers, emulsifiers, stabilizers, buffers, and others, as appropriate. Examples of these excipients are isostearylstearate, isopropyl myristate, glycerin, mineral oil, polycarbophil, carbomer 934P or 940, hydrogenated palm oil, glyceride, sodium hydroxide, sorbic acid, and purified water.

One formulation has between about 0.01-10%, by weight, of each of the active agents, about 60-90%, by weight, lipophilic carrier, between about 0.1-25%, by weight, mucoadhesive agent, between about 1-25%, by weight, sorption promoter and optionally a penetration enhancer or solubilizing agent, usually present in 1-30%, by weight.

The composition for transmucosal delivery is administered either directly to the vagina or is incorporated into the intravaginal device.

The intravaginal device of the invention may be a tampon, tampon-like device, ring, pessary, strip, cup or foam which has a solid structure into which the formulation is incorporated and from which it is released in a timely fashion over a period of time.

The intravaginal device for vaginal or transmucosal vaginal delivery may also be an intravaginal tampon, intravaginal ring, intravaginal pessary, intravaginal sponge, intravaginal tablet or other intravaginal device.

As stated above, the method of this form of the present invention is anticipated to be suitable for promoting implantation.

Accordingly, in another embodiment the present invention provides a method of promoting implantation of an embryo into uterine endometrium in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

In one embodiment, the exposure occurs from ovulation to mid-pregnancy.

In addition, this form of the present invention is also anticipated to be suitable for the production of an intravaginal composition for promoting implantation of an embryo into uterine endometrium in a subject.

Accordingly, in another embodiment the present invention provides an intravaginal composition for promoting implantation of an embryo into uterine endometrium in a subject, the composition including IGF-II, or a variant or an analogue thereof; and either or both of plasminogen, or a variant or an analogue thereof, and urokinase plasminogen activator, or a variant or an analogue thereof.

The present invention is also anticipated to be suitable for reducing the likelihood of implantation failure embryo.

Accordingly, in another embodiment the present invention provides a method of reducing the likelihood of failure of an embryo to implant into uterine endometrium in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

In one embodiment, the exposure occurs from ovulation to mid-pregnancy.

In addition, this form of the present invention is also anticipated to be suitable for the production of an intravaginal composition for reducing the likelihood of implantation failure of an embryo in a subject.

Accordingly, in another embodiment the present invention provides an intravaginal composition for reducing the likelihood of failure of an embryo to implant into uterine endometrium in a subject, the composition including IGF-II, or a variant or an analogue thereof; and either or both of plasminogen, or a variant or an analogue thereof, and urokinase plasminogen activator, or a variant or an analogue thereof.

The present invention is also anticipated to be suitable for promoting placental development in a subject.

Accordingly, in another form the present invention provides a method of promoting placental development in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

In one embodiment, the exposure occurs from ovulation to mid-pregnancy.

The present invention is also anticipated to be suitable for the production of an intravaginal composition for promoting placental development.

Accordingly, in another embodiment the present invention provides an intravaginal composition for promoting placental development in a subject, the composition including IGF-II, or a variant or an analogue thereof and either or both of plasminogen, or a variant or an analogue thereof, and urokinase plasminogen activator, or a variant or an analogue thereof.

The present invention is also anticipated to be suitable for improving placental growth and/or function in a subject.

Accordingly, in another embodiment the present invention provides a method of improving placental growth and/or function in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

In one embodiment, the exposure occurs from ovulation to mid-pregnancy.

The present invention is also anticipated to be suitable for the production of an intravaginal composition for improving placental growth and/or function.

Accordingly, in another embodiment the present invention provides an intravaginal composition for improving placental growth and/or function in a subject, the composition including IGF-II, or a variant or an analogue thereof; and either or both of plasminogen, or a variant or an analogue thereof, and urokinase plasminogen activator, or a variant or an analogue thereof.

The present invention is also anticipated to be suitable for reducing the likelihood of miscarriage occurring in a subject by use of an intravaginal composition.

Accordingly, in another embodiment the present invention provides a method of reducing the likelihood of miscarriage occurring in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

In one embodiment, the exposure occurs from ovulation to mid-pregnancy.

The present invention also provides an intravaginal composition that is anticipated to reduce the likelihood of miscarriage occurring in a subject.

Accordingly, in another embodiment the present invention provides an intravaginal composition for reducing the likelihood of miscarriage occurring in a subject, the composition including IGF-II, or a variant or an analogue thereof; and either or both of plasminogen, or a variant or an analogue thereof, and urokinase plasminogen activator, or a variant or an analogue thereof.

The present invention is also anticipated to be suitable for reducing the extent and/or likelihood of pre-eclampsia, occurring in a subject by use of an intravaginal composition.

Accordingly, in another embodiment the present invention provides a method of reducing the extent and/or likelihood of pre-eclampsia occurring in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

The present invention also provides an intravaginal composition that is anticipated to reduce the extent and/or likelihood of pre-eclampsia occurring in a subject.

Accordingly, in another embodiment the present invention provides an intravaginal composition for reducing the extent and/or likelihood of pre-eclampsia occurring in a subject, the composition including IGF-II, or a variant or an analogue thereof; and either or both of plasminogen, or a variant or an analogue thereof, and urokinase plasminogen activator, or a variant or an analogue thereof.

The present invention is also anticipated to be suitable for reducing the extent and/or likelihood of intrauterine growth restriction occurring in a subject by use of an intravaginal composition.

Accordingly, in another embodiment the present invention provides a method of reducing the extent and/or likelihood of intrauterine growth restriction occurring in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

In one embodiment, the exposure occurs from ovulation to mid-pregnancy.

The present invention also provides an intravaginal composition that is anticipated to reduce the extent and/or likelihood of intrauterine growth restriction (fetal growth restriction) occurring in a subject.

Accordingly, in another embodiment the present invention provides an intravaginal composition for reducing the extent and/or likelihood of intrauterine growth restriction occurring in a subject, the composition including IGF-II, or a variant or an analogue thereof; and either or both of plasminogen, or a variant or an analogue thereof, and urokinase plasminogen activator, or a variant or an analogue thereof.

The present invention is also anticipated to be suitable for reducing the likelihood of pre-term labour occurring in a subject by use of an intravaginal composition.

Accordingly, in another embodiment the present invention provides a method of reducing the likelihood of pre-term labour occurring in a subject, the method including the step of intravaginally exposing the subject to an effective amount of IGF-II, or a variant or an analogue thereof the method further including the step of intravaginally exposing the subject to either or both of an effective amount of plasminogen, or a variant or analogue thereof, and an effective amount of urokinase plasminogen activator, or a variant or an analogue thereof.

In one embodiment, the exposure occurs from ovulation to mid-pregnancy.

The present invention also provides an intravaginal composition that is anticipated to reduce the likelihood of pre-term labour occurring in a subject.

Accordingly, in another embodiment the present invention provides an intravaginal composition for reducing the likelihood of pre-term labour occurring in a subject, the composition including including IGF-II, or a variant or an analogue thereof; and either or both of plasminogen, or a variant or an analogue thereof, and urokinase plasminogen activator, or a variant or an analogue thereof.

The present invention is also anticipated to be suitable for normalising the gestation period of a fetus carried by a subject by use of an intravaginal composition.

Accordingly, in another embodiment the present invention provides a method of normalising the length of the gestation period of a fetus carried by a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

In one embodiment, the exposure occurs from ovulation to mid-pregnancy.

The present invention also provides an intravaginal composition that is anticipated to increase the likelihood that a fetus is carried to term or near term in a subject.

Accordingly, in another embodiment the present invention provides an intravaginal composition for increasing the likelihood that a fetus is carried to term or near term in a subject, the composition including IGF-II, or a variant or an analogue thereof; and either or both of plasminogen, or a variant or an analogue thereof, and urokinase plasminogen activator, or a variant or an analogue thereof.

The present invention is also anticipated to be suitable for reducing the extent and/or likelihood of placental abruption occurring in a subject by use of an intravaginal composition.

Accordingly, in another embodiment the present invention provides a method of reducing the extent and/or likelihood of placental abruption occurring in a subject, the method including the step of intravaginally exposing the subject to an effective amount of IGF-II, or a variant or an analogue thereof, the method further including the step of intravaginally exposing the subject to either or both of an effective amount of plasminogen, or a variant or analogue thereof, and an effective amount of urokinase plasminogen activator, or a variant or an analogue thereof.

In one embodiment, the exposure occurs from ovulation to mid-pregnancy.

The present invention also provides an intravaginal composition that is anticipated to reduce the extent and/or likelihood of placental abruption occurring in a subject.

Accordingly, in another embodiment the present invention provides an intravaginal composition for reducing the extent and/or likelihood of placental abruption occurring in a subject, the composition including IGF-II, or a variant or an analogue thereof; and either or both of plasminogen, or a variant or an analogue thereof, and urokinase plasminogen activator, or a variant or an analogue thereof.

The present invention also provides methods for improving post-natal outcome due to improved placental development and/or a reduction in the risk and/or likelihood of pregnancy complications due to deficient placental development, by intravaginal exposure as described above. The present invention also provides compositions and combination products to improve post-natal outcomes.

### DESCRIPTION OF SPECIFIC EMBODIMENTS

Reference will now be made to experiments that embody the above general principles of the present invention. However, it is to be understood that the following description is not to limit the generality of the above description.

### Example 1

### Media

Suitable base media generally include HTF medium, Whittinghams T6 medium, Hams F10, Earles solution, IVF50 (Scandinavian IVF Science), S2 (Scandinavian IVF Science), G1.2 (Scandinavian IVF Science) and G2.2 (Scandinavian IVF Science).

A suitable medium is as follows:

| Working pH range | 7.2-7.4 | |
|---|---|---|
| Components | Mg/L | |
| IGF-II | 1-750 | nM |
| Plasminogen | 0.01-50 | μg/ml |

Inorganic Salts

| | |
|---|---|
| CaCl₂—2H₂O | 250.00 |
| KCL | 186.38 |
| KH₂PO₄ | 47.99 |
| MgSO₄ | — |
| MgSO₄7H₂O | 49.30 |
| NaCl | 5551.80 |
| NaHCO₃ | 2100.25 |

Other Components

| | |
|---|---|
| EDTA | 3.72 |
| D-Glucose | 36.03 |
| Sodium Lactate | 1121.00 |
| Lactate NaSalt (ml/L) 1.42 | — |
| Sodium Pyruvate | 22.00 |
| HSA | 1000.00 |
| Phenol Red | — |

Amino Acids

| | |
|---|---|
| L-Arginine | 63.20 |
| L-Cystine | 12.02 |
| L-Cystine-2HCL | — |
| L-Glutamine | 146.15 |
| Glycine | 3.75 |
| L-Histidine | — |
| L-Histidine•HCl•H₂O | 20.96 |
| L-Isoleucine | 26.23 |
| L-Leucine | 26.24 |
| L-Lysine | — |
| L-Lysine•HCl | 36.52 |
| L-Methionine | 7.46 |
| L-Phenylalanine | 16.52 |
| L-Serine | 5.26 |
| L-Threonine | 23.82 |
| L-Tryptophan | 5.11 |
| L-Tyrosine | 18.12 |
| L-Tyrosine NaH₂O | — |
| L-Valine | 23.42 |
| L-Alanine | 4.45 |
| L-Asparagine | — |
| L-Asparagine-H₂O | 7.50 |
| L-Aspartic Acid | 6.66 |
| L-Glutamic Acid | 7.36 |
| L-Proline | 5.76 |

Antibiotics

| | |
|---|---|
| Pen G Na Salt (units) | 100,000.00 |
| Strep Sulfate | 50.00 |

Medium M1:

| | Concentration in medium (mM) |
|---|---|
| Chemicals | |
| NaCl | 22-550 |
| K₂SO₄ | 1-27 |
| NaH₂PO₄, anhyd | 0.2-5 |
| Ca-lactate | 0.7-18 |
| MgSO₄, anhyd | 0.16-4 |
| D-Glucose | 0.1-2.5 |
| Na-Pyruvat | 0.04-1 |
| L-Glutamine | 0.06-1.5 |
| Taurine | 0.02-0.5 |
| HEPES, Acid | 0.5-12.5 |
| Phenol Red 0.5% | 0.2 ml-5.0 ml per 2 litres |
| Non- Ess.- AA | |
| L-Alanine | 0.02-0.5 |
| L-Asparagine | 0.02-0.5 |
| L-Aspartic Acid | 0.02-0.5 |
| L-Glutamic Acid | 0.02-0.5 |
| Glycine | 0.02-0.5 |
| L-Proline | 0.02-0.5 |
| L-Serine | 0.02-0.5 |
| NaHCO₃ | 5-125 |
| Pen/strep × 1000 | 0.4-10 ml per 2 litres |
| Comp 1 | 0.4-10 ml per 2 litres |
| Comp 2 | 0.4-10 ml per 2 litres |
| EDTA* | 10 μm-50 μm |
| HSA, 20% | 4-100 ml per 2 litres |

Medium M2:

| | Concentration in medium (mM) |
|---|---|
| Chemicals | |
| NaCl | 22-550 |
| K₂SO₄ | 1-27 |
| NaH₂PO₄, anhyd | 0.2-5 |
| Ca-lactate | 0.7-18 |
| MgSO₄, anhyd | 0.16-4 |
| NaHCO₃ | 5-130 |
| D-Glucose | 0.2-5 |
| Na-Pyruvat | 0.02-0.5 |
| L-Glutamine | 0.2-5 |
| Phenol Red | 0.2-5 ml per 2 litres |
| Non-Ess-AA | |
| L-Alanine | 0.02-0.5 |
| L-Asparagine | 0.02-0.5 |
| L-Aspartic Acid | 0.02-0.5 |
| L-Glutamic Acid | 0.02-0.5 |
| Glycine | 0.02-0.5 |
| L-Proline | 0.02-0.5 |
| L-Serine | 0.02-0.5 |
| Ess-AA | |
| L-Arginine | 0.6-15 |
| L-Cystine | 0.25-6.25 |
| L-Histidine | 0.25-6.5 |
| L-Isoleucine | 1-25 |
| L-Leucine | 1-25 |
| L-Lysine HCl | 0.8-20 |
| L-Methionine | 0.25-6.5 |
| L-Phenylalanine | 0.5-12.5 |
| L-Threonine | 1-25 |
| L-Tryptophan | 0.2-25 |
| L-Tyrosine | 0.5-12.5 |
| L-Valine | 1-25 |
| Pen/strep × 1000 | 0.4-10 ml per 2 litres |
| EDTA* | 1 μm-25 μm |
| Comp. 1 | 0.4-10 ml per 2 litres |
| Comp. 2 | 0.4-10 ml per 2 litres |
| HSA, 20% | 4-100 ml per 2 litres |

Comp. 1 consists of Milli RX Water, C₆H₅Na₃O₇*2H₂O, Citric acid monohydrate, Pluronic F-68, Aurintricarboxylic Acid, Ethylenediaminetetraacetic acid, Ethylendiaminetetraacetic dina, and Trace Elements.
Comp. 2 consists of Milli RX Water, Saltsyre 1 N, HCL, and Human Insulin Recombinant.

### Example 2

### Culturing of Embryos

Ten female C57/B16 mice aged 3 weeks were obtained each week from the University of Adelaide Medical School Animal House and maintained at 22-23° C., on a cycle of 12 hours light, 12 hours dark. Mice received food and water ad libitum. At 3 pm on day −3 they were given 5 IU equine chorionic gonadotrophin (eCG, Folligon) i.p. At 3 pm on day—1 mice were injected with 5 IU hCG (Chorulon) i.p. On the evening of day—1 mice were placed with separately housed stud Balb/c male mice and checked for a vaginal copulatory plug the next morning and females were removed from males. The day of plug detection was designated day 1 of embryo development. Females were killed by cervical dislocation at 2:00 pm on this day. Oviducts were excised and placed in Medium M1 media (Medicult A/s, Denmark) at 37° C. Embryos were flushed from the oviducts using the same media and then rinsed four times in 100 μl drops of embryo culture media.

Embryos were placed in 20, 25 or 50 μl drops of Medium M1 media (Medicult A/s, Denmark) in 3 cm petri dishes and overlain with embryo tested mineral oil (Sigma). 10-15 embryos were plated per drop. Embryos were cultured until day 3 of embryo development at 37° C., in 5% CO₂ and 5% O₂. Embryos were scored on day 2 and day 3 at which time they were transferred to fresh Medium M2 media (Medicult A/s, Denmark) and cultured until day 5 of embryo development. Embryos were cultured with 0-100 nM receptor grade IGF-II (GroPep, Adelaide, SA) or 10 μg/ml plasminogen (Sigma) or 5 μg/ml urokinase plasminogen activator (uPA) (Sigma).

Embryos were scored according to stage of development; 1-cell, 2-cell, 4-8 cell, morula and blastocyst. The number of 2-cell embryos progressing to each subsequent stage by day 5 were counted.

Initially, to establish that the small amount of acetic acid present in rhIGF-II samples did not interfere with embryo development, we added 1 μl 0.1 M filter sterile acetic acid to 999 μl Medium M1 and M2 media and cultured 1-cell embryos in groups of 10 in 20 μl drops sequentially as above and compared embryo development in Medium M1 and M2 media only. There were 3 replicates of 4 drops per treatment to a total of 120 embryos per treatment. There was no difference in the development of embryos from 1-cell to the blastocyst stage between media plus acetic acid or media alone groups.

To establish that medium with IGF-II supported embryonic development, embryos were cultured in Medium M1 and M2 media as above with or without different doses of rhIGF-II (0, 0.5, 1, 12.5, 25 or 50 μM rhIGF-II (GroPep Pty Ltd, Adelaide); or 0.5, 1, 12.5, 25, 50 or 100 nM rhIGF-II (GroPep Pty Ltd, Adelaide). There were 4 replicates of the nanomolar range of IGF-II treatments and 6 replicates of the picomolar IGF-II treatments; 6 groups with 40 in each to a total of 160-240 embryos per treatment. It was found that the addition of IGF-II at all concentrations supported the development of embryos from 1-cell to blastocyst stages.

To establish that the addition of plasminogen and uPA to the Medium M1 and M2 media supports growth of embryos, we cultured 1 replicate of 40-50 1-cell embryos in Medium M1 and M2 media alone as above, or in Medium M1 and M2 media with 10 μg/ml plasminogen (Sigma) or with 5 μg/ml uPA (Sigma) or 0.5 μM rhIGF-II (GroPep Pty Ltd). In this particular replicate, although only 37% of control embryos in media alone developed to the blastocyst stage, 64% of embryos cultured with 10 g/ml plasminogen, 56% of embryos cultured with 0.5 μM rhIGF-II and 59% of embryos cultured with 5 μg/ml uPA developed to the blastocyst stage.

Thus, the addition of these compounds appears to have rescued embryos that would otherwise have died and/or increased embryo viability.

Accordingly, a medium including IGF-II and either or both of plasminogen and uPA is anticipated to support embryonic development. It is further anticipated that a medium including all three components will be particularly effective.

In addition, while these experiments have been performed on embryos from mice, it is also anticipated that these results will also apply to embryos from other mammalian species, and in particular, will be applicable to the culturing of human embryos.

### Example 3

### IGF-II Appears to Promote Blastomere Proliferation and Survival

To establish that the addition of IGF-II to the Medium M1 and M2 media improves embryo development by increasing total blastocyst cell number on day 5 of development, we cultured 2 replicates of 1-cell embryos in Medium M1 and M2 media with 0, 0.5, 1, 12.5, 25 or 50 μM rhIGF-II 2 to a total of 10-25 embryos in each group. On day 5 of development, blastocysts were stained with bisbenzimide to label nuclei which were counted using a fluorescence microscope. The addition of picomolar concentrations of rhIGF-II to the media increased the total number of cells in the blastocysts by 23% compared to controls cultured in media alone. This indicates that IGF-II is likely to promote blastomere (cells of the embryo) proliferation and survival and thereby would enhance the quality and viability of embryos generated by IVF.

It is understood in the art that better quality embryos will have a greater capacity to implant and invade into the uterine endometrium and thereby improve pregnancy outcome.

Therefore the combination of IGF-II with either or both of plasminogen and uPA would be expected to promote blastocyst growth, survival, hatching, implantation and trophoblast invasion. These are key to the embryo initiating placental morphogenesis and any defect at this point is absolutely deleterious to the pregnancy.

Finally, it has been previously shown that the fetus and placenta generated from blastocysts with greater cell numbers are larger. Accordingly, this may be expected to reduce the chance of intrauterine growth restriction (IUGR).

### Example 4

### IVF Medium

IVF Medium for fertilisation of human oocytes and culture of embryos up to the 4-8 cell stage (Day 2 and 3 after insemination). This IVF Medium includes 0.0003 to 750 ng/ml IGF-II, and either or both of 0.01 to 50 μg/ml plasminogen, and 0.01 to 50 μg/ml urokinase plasminogen activator, and can also be used for embryo transfer.

### Composition

Earle's Balanced Salt Solution (EBSS) supplemented with:
Synthetic Serum Replacement (SSR®)
Human serum albumin (HSA)
Glucose
Sodium pyruvate
Sodium bicarbonate
Streptomycin 50 mg/litre
Penicillin 50,000 IU/litre

### Use

1. After collection, the oocytes are individually transferred into sterile four-well culture dishes, each well containing 0.5 ml of IVF Medium equilibrated in the CO₂ incubator with 5% O₂ overnight.
2. In open culture systems between 50 and 100,000 motile spermatozoa are added to each oocyte approximately 5-6 hours after oocyte retrieval.
3. After approximately 18 hours incubation in the presence of spermatozoa at 37° C. in 5% CO₂, 5% O₂ and humidified air, the oocytes are examined microscopically for the presence of two pronuclei, indicating that normal fertilisation has occurred. The oocytes are then transferred into new four-well culture dishes or microdrops of media.
4. The resulting embryos are replaced in the uterus 2-3 days after oocyte retrieval when they have reached 4-8 cell stage using IVF Medium as the transfer medium.

### Example 5

### Blastocyst Development

This Example describes use of IGF-II and either or both of plasminogen and urokinase plasminogen activator from fertilisation through to blastocyst development.

### Composition

### Medium 1

0.0003 to 750 ng/ml IGF-II
0.01 to 50 μg/ml plasminogen
0.01 to 50 μg/ml urokinase plasminogen activator

### Synthetic Serum Replacement (SSR®) (USA Art Supplement)

Human serum albumin (HSA)

### Glucose

Sodium pyruvate

### Lactate

Potassium sulphate
Magnesium sulphate
Sodium chloride
Sodium hydrogen phosphate

### Non-essential Amino Acids

### L-glutamine

### Taurine

Sodium bicarbonate

### HEPES

Streptomycin 50 mg/litre

### Penicillin 50,000 IU/litre

### Phenol Red

### Medium 2

0.0003 to 750 ng/ml IGF-II
0.01 to 50 μg/ml plasminogen
0.01 to 50 μg/ml urokinase plasminogen activator

### Synthetic Serum Replacement (SSR®) (USA=Art Supplement)

Human serum albumin (HSA)

### Glucose

Sodium pyruvate

### Lactic Acid

Potassium sulphate
Magnesium sulphate
Sodium hydrogen phosphate

### Essential Amino Acids

### Non-essential Amino Acids

### L-glutamine

Sodium bicarbonate
Streptomycin 50 mg/litre

### Penicillin 50,000 IU/litre

### Use

Two protocols may be used:

### Protocol 1:

### Day 5 Embryo Transfer

1. Recover oocytes as normal and prepare sperm according to preferred procedure. Carry out fertilisation in Medium 1 and where ICSI is required, transfer to Medium 1 immediately after injection.
2. At 16-20 hours, check for pronuclei, then carefully wash and transfer zygotes to fresh microdrops or open dishes of Medium 1. Culture volumes of 50 μl for microdrops and 0.5 mls for wells/dishes should be used and embryos may be cultured singly or in multiples to a maximum of 4 embryos.
3. At the 4-8 cell stage, the embryos should be carefully washed in Medium 2 and transferred to fresh microdrops or 0.5 mls for wells/dishes should be used and the embryos should be cultured singly or in multiples to a maximum of 4 in this second stage medium.
4. The embryos should be moved to fresh drops of Medium 2 every other day until blastocyst formation at approximately Day 5.
5. Embryos should be prepared and transferred to the uterus in Medium 2.

### Protocol 2:

### Procedure for Day 2 or Day 5 Embryo Transfer

If enough oocytes or zygotes are available, the embryologist may elect to split the oocytes or zygotes between both media types where the couple's blastocyst formation potential is unknown, in order to increase the chances of having an embryo transfer. Zygotes can either be divided for Day 2 transfer or freezing at the pronuclear/cleavage stage and ‘Medium 1 and 2’ for development only to blastocyst. It is not recommended to transfer embryos cultured in the latter formulations on Day 2.
1. Recover oocytes as normal and prepare sperm according to preferred procedure. Carry out fertilisation in IVF medium and where ICSI is required, transfer to IVF medium immediately after injection.
2. When checking for pronuclei between 16-20 hours, zygotes intended to be cultured to Day 2 for replacement/cryopreservation, should be transferred to IVF medium and cultured in this medium until replacement/cryopreservation. Those embryos cultured in IVF can be frozen using Embryo Freezing media products.
3. Zygotes intended to be cultured to the blastocyst stage, should be washed carefully in Medium 1 and then transferred to fresh microdrops or dishes/wells of the same medium. Culture volumes of 50 μl for microdrops and 0.5 mls for open dishes should be used and embryos may be cultured singly or in multiples to a maximum of 4 embryos.
4. At the 4-8 cell stage, embryos in Medium 1 should be carefully washed in Medium 2 and then transferred to fresh microdrops or open dishes of the same medium. Culture volumes of 50 μl for microdrops and 0.5 mls for wells/dishes should be used and embryos can be cultured singly or in multiples to a maximum of 4 in this second stage medium.
5. The embryos should be moved to fresh drops of Medium 2 every other day until blastocyst formation occurs at approximately Day 5.

Embryos should be prepared and transferred to the uterus in Medium 2.

### Example 6

### Culture of Embryos from Day 3

This Example describes the extended culture of embryos from Day 3 and may also be used for embryo transfer.

### Composition

M3 Medium is based on MCDB 302; a modification of Ham's F10 and F12 composed of 0.0003 to 750 ng/ml IGF-II, 0.01 to 50 μg/ml plasminogen, 0.01 to 50 μg/ml urokinase plasminogen activator, amino acids, vitamins, inorganic salts and glucose supplemented with:
Synthetic Serum Replacement (SSR®) (USA-Art Supplement)
Human serum albumin (HSA)
Sodium bicarbonate
Streptomycin 25 mg/litre
Penicillin 25,000 IU/litre

### Use

1. After 2 days of culture (approximately 48 hours after oocyte recovery) fertilised and normally cleaved embryos are transferred into new four-well culture dishes.
2. Each well contains 0.5 ml of M3 Medium, which has been equilibrated in the CO₂ incubator with 5% O₂ overnight before use. Each embryo is cultured separately in its own well, making the selection of embryos for replacement easier.
3. A minimum of 24 h or culture in M3 Medium should be used before replacement.

### Example 7

### M1, M2 and UM

M1 can be used both for the IVF insemination procedure and for culture of embryos until Day 3.

M2 is for culture of embryos from Day 3 and through to the blastocyst stage. UM is especially designed for transfer of embryos cultured in M1 or M2.

### Composition

M1 and M2, both include 0.0003 to 750 ng/ml IGF-II, 0.01 to 50 μg/ml plasminogen and 0.01 to 50 μg/ml urokinase plasminogen activator:

Human serum albumin (HSA)
Glucose and derived metabolites
Physiological salts
Essential amino acids
Non-essential amino acids

### Vitamins

### Nucleotides

Sodium bicarbonate
Streptomycin 40 mg/litre

### Penicillin 40,000 IU/litre

UM, including 0.0003 to 750 ng/ml IGF-II, 0.01 to 50 μg/ml plasminogen, and 0.01 to 50 μg/ml urokinase plasminogen activator
Sodium hyaluronate Ph. Eur.4Ed.
Human serum albumin (HSA)
Glucose and derived metabolites
Physiological salts
Essential amino acids
Non-essential amino acids

### Vitamins

### Nucleotides

Sodium bicarbonate
Streptomycin 40 mg/litre

### Penicillin 40,000 IU/litre

### Use

1. Recover oocytes as normal and prepare sperm according to preferred procedure. Carry out insemination (Day 0) in pre-equilibrated M1.
2. a) If IVF Medium was chosen as the insemination medium for IVF, in order to get better embryo morphology an early rinse in M1 is recommended at 4 hours, after which the oocytes are carefully transferred to a pre-equilibrated culture dish containing 50 μl microdrops or 0.5 ml wells/dishes of M1 covered with Liquid Paraffin.
   b) In case of ICS1, immediately after injection the oocytes are transferred to the pre-equilibrated culture dish of M1.
3. At 16-20 hours (Day 1), check for formation of pronuclei, then carefully wash and transfer zygotes to fresh 50 μl microdrops or 0.5 ml wells/dishes of M1 covered with Liquid Paraffin. The embryos should be cultured singly or in multiples to a maximum of 4 per well and are kept in the same medium until Day 3.

### Embryo Transfer Day 2 or Day 3:

The embryos are prepared and transferred to the uterus in 20 to 30 μl of pre-equilibrated UM.

At Day 3, in case of blastocyst culture, the embryos are carefully washed in pre-equilibrated M2 and transferred to fresh 50 μl microdrops or 0.5 ml wells/dishes of the same medium. The embryos should be cultured singly or in multiples to a maximum of 4 per well in this second stage medium, until blastocyst formation at approximately Day 5. embryo transfer media should have IGF-II, and either or both plasminogen and uPA.

### Blastocyst Transfer:

Blastocysts should be prepared and transferred to the uterus in 20 to 30 μl of pre-equilibrated UM.

### Example 8

### Cream Formulation for Intravaginal Exposure

A cream formulation may be prepared using includes the following components:

| IGF-II | 1 to 20 | mg |
|---|---|---|
| Plasminogen | 0.1 to 10 | mg |
| uPA | 0.1 to 10 | mg |
| Beeswax | 2.7 | g |
| Carbopol ® 934 q.s. | 100.0 | g |

Mixing is conducted with tile and spatula until a homogeneous cream mixture is obtained having the active components uniformly dispersed throughout the composition.

### Example 9

### Cream Formulation for Intravaginal Exposure

The procedure of Example 5 may be repeated except that the following components may be used:

| IGF-II | 1 to 20 | mg |
|---|---|---|
| Plasminogen | 0.1 to 10 | mg |
| uPA | 0.1 to 10 | mg |
| Polyethylene glycol 400 | 37.5 | g |
| 1,2,6-hexanetriol | 20.0 | g |
| Polyethylene glycol 4000 q.s. | 100.0 | g |

A homogenous cream mixture will be obtained.

### Example 10

### Cream Formulation for Intravaginal Exposure

The procedure of Example 5 is repeated except that the following components may be used:

| IGF-II | 1 to 20 | mg |
|---|---|---|
| Plasminogen | 0.1 to 10 | mg |
| uPA | 0.1 to 10 | mg |
| Polyethylene glycol 400 | 37.5 | g |
| Polyethethylene glycol 400 monostearate | 26.0 | g |
| Polyethylene glycol 4000 q.s. | 100.0 | g |

A homogenous cream mixture will be obtained.

### Example 11

### Cream Formulation for Intravaginal Exposure

The procedure of Example 5 is repeated except that the following components may be used:

| IGF-II | 1 to 20 | mg |
|---|---|---|
| Plasminogen | 0.1 to 10 | mg |
| uPA | 0.1 to 10 | mg |
| Polyethylene glycol 400 | 47.5 | g |
| Cetyl Alcohol | 5.0 | g |
| Polyethylene glycol 4000 q.s. | 100.0 | g |

A homogenous cream mixture will be obtained.

### Example 12

### Ointment Formulation for Intravaginal Exposure

An ointment formulation may be prepared using the following components:

| IGF-II | 1 to 20 | mg |
|---|---|---|
| Plasminogen | 0.1 to 10 | mg |
| uPA | 0.1 to 10 | mg |
| Anhydrous lanolin | 20.0 | g |
| Mineral Oil | 25.0 | g |
| White Petrolatum q.s. | 100.0 | g |

Mixing is conducted with tile and spatula until a homogeneous ointment mixture is obtained having the active components uniformly dispersed throughout the composition.

### Example 13

The procedure of Example 5 is repeated except that the following components may be used:

| IGF-II | 1 to 20 | mg |
|---|---|---|
| Plasminogen | 0.1 to 10 | mg |
| uPA | 0.1 to 10 | mg |
| Diisopropyl Adipate | 19.95 | g |
| White Petrolatum USP q.s. | 100.0 | g |

A homogenous ointment mixture will be obtained.

### Example 14

### Further Additives to Cream and Ointment Formulations

In the cream and ointment formulations described in Examples 5-10, optional ingredients can include materials such as antioxidants, viscosity modifiers (e.g., paraffin wax or lanolin wax), and topical absorption rate modifiers.

Finally, it will be appreciated that various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the present invention.

1-78. (canceled)

79. An oocyte and/or embryo culture medium, the medium including 0.0003 to 750 ng/ml IGF-II, or a variant or analogue thereof, the medium further including either or both of 0.01 to 50 μg/ml plasminogen, or a variant or analogue thereof, and 0.01 to 50 μg/ml urokinase plasminogen activator, or a variant or analogue thereof.

80. A medium according to claim 79, wherein the medium includes 0.003-375 ng/ml IGF-II.

81. A medium according to claim 79, wherein the medium includes 1 to 20 μg/ml plasminogen.

82. A medium according to claim 79, wherein the medium includes 1 to 20 μg/ml urokinase plasminogen activator.

83. A medium according to claim 79, wherein the medium is used for one or more of the following purposes: in vitro maturation of an oocyte; culturing an early stage embryo to blastocyst stage; transfer of an embryo to a subject; promoting implantation of an embryo into uterine endometrium; promoting embryo development; improving placental development; improving placental growth and/or function; reducing the likelihood of failure of an embryo to implant into uterine endometrium in a subject; promoting placental development in a subject; reducing the likelihood of miscarriage occurring in a subject; reducing the extent and/or likelihood of pre-eclampsia occurring in a subject; reducing the extent and/or likelihood of intrauterine growth restriction occurring in a subject; reducing the likelihood of pre-term labour occurring in a subject; increasing the likelihood that a fetus is carried to term or near term in a subject; reducing the extent and/or likelihood of placental abruption occurring in a subject; and improving post-natal outcome due to improved placental development and/or a reduction in the risk and/or likelihood of pregnancy complications due to deficient placental development.

84. A combination product including the following components:
an oocyte and/or embryo culture medium;
IGF-II, or a variant or an analogue thereof, and either or both of plasminogen, or a variant or analogue thereof; and
urokinase plasminogen activator, or a variant or an analogue thereof;
wherein the components are provided in a form for addition of IGF-II, and either or both of plasminogen and urokinase plasminogen activator, to the culture medium so as to produce a culture medium including 0.0003 to 750 ng/ml IGF-II (or a variant or an analogue thereof), and either or both of 0.01 to 50 μg/ml plasminogen (or a variant or an analogue thereof) and 0.01 to 50 μg/ml urokinase plasminogen activator (or a variant or an analogue thereof).

85. A method of culturing an oocyte and/or embryo, the method including culturing the oocyte and/or embryo in a medium according to claim 79.

86. A method of assisted reproduction involving an oocyte or embryo, the method including culturing the oocyte or embryo in a medium according to claim 79.

87. A method according to claim 86, wherein the method of assisted reproduction is in vitro fertilization.

88. A method of promoting development of an embryo, the method including culturing the embryo in a medium according to claim 79 and/or culturing an oocyte used to produce the embryo in a medium according to claim 79.

89. A method of maturing an oocyte, the method including culturing the oocyte in a medium according to claim 79.

90. A method of promoting implantation of an embryo into uterine endometrium, the method including culturing the embryo in a medium according to claim 79 and/or culturing an oocyte used to produce the embryo in a medium according to claim 79.

91. A method of improving placental development for an isolated embryo implanted into uterine endometrium, the method including culturing the embryo in a medium according to claim 79 and/or culturing an oocyte used to produce the embryo in a medium according to claim 79.

92. A method of improving placental growth and/or function for an embryo implanted into uterine endometrium, the method including culturing the embryo in a medium according to claim 79 and/or culturing an oocyte used to produce the embryo in a medium according to claim 79.

93. A method of reducing the extent and/or likelihood of intrauterine growth restriction in a subject with an isolated embryo introduced into the subject, the method including culturing the embryo in a medium according to claim 79 and/or culturing an oocyte used to produce the embryo in a medium according to claim 79.

94. A method of reducing the likelihood of pre-term delivery of a fetus produced from an isolated embryo introduced into a subject, the method including culturing the embryo in a medium according to claim 79 and/or culturing an oocyte used to produce the embryo in a medium according to claim 79.

95. A method of increasing the likelihood that a fetus is carried to term or near term in a subject, the fetus being produced from an isolated embryo introduced into the subject, the method including culturing the embryo in a medium according to claim 79 and/or culturing an oocyte used to produce the embryo in a medium according to claim 79.

96. A method of increasing the length of the gestation period of a fetus carried by a subject, the fetus being produced from an isolated embryo introduced into the subject, the method including culturing the embryo in a medium according to claim 79 and/or culturing an oocyte used to produce the embryo in a medium according to claim 79.

97. A method of reducing the extent and/or likelihood of abruption of a placenta formed by implantation of an isolated embryo into uterine endometrium, the method including culturing the embryo in a medium according to claim 79 and/or culturing an oocyte used to produce the embryo in a medium according to claim 79.
